SEQUENCE LISTING (38 sequences)

SEQ ID NO: 1 - DNA, 798 bp, Artificial Sequence
  Synthetic single-chain variable fragment
atggccctgcccgtgaccgctctcctgctgcctctggccctgctcctccatgctgccagacccgacatccagatgacacagacaaccagcagcctgtccgcttccctcggagacagggtgacaatttcctgcagggccagccaggacatcagcaagtacctgaactggtaccagcagaaacccgacggcaccgtcaagctcctgatctaccacaccagcagactgcacagcggagtgccttccaggttcagcggcagcggctccggcaccgattactccctgaccattagcaacttagaacaggaggacattgccacctacttttgtcagcagggcaacaccctcccctacacctttggaggcggaaccaagttagaaatcaccggcggcggcggcagcggaggaggaggcagcggaggcggaggctccgaggtgaaactgcaggagagcggccccggactggtcgcccctagccaatccctctccgtcacctgcaccgtgagcggagtgagcctgcctgactacggagtgagctggatcagacagccccctaggaaaggactggaatggctgggcgtgatttggggcagcgagaccacctattacaacagcgccctgaagtccagactgacaatcatcaaggacaatagcaaaagccaagtgtttctgaagatgaacagcctgcagaccgatgacaccgccatctattattgcgccaagcactactactacggaggaagctacgctatggattattggggccaaggcacaagcgtgaccgtcagcagcgcggccgcc

SEQ ID NO: 2 - DNA, 921 bp, Artificial Sequence
  Synthetic single-chain variable fragment
atggccttaccagtgaccgccttgctcctgccgctggccttgctgctccacgccgccaggccgatggaaaaggatacactgttgttgtgggttctgctcctgtgggtgcccggcagcaccggagatattgtgctgacgcagtctcctgcatcactcgccgtgtctctgggccagcgcgctaccatcagctgcagagcctctgaaagtgttgacaattatggaatttctttcatgaattggttccagcagaagcctggccagcccccgaaactcctcatatatgccgcgtctaatcagggctctggggtccctgctagattttctggcagcggctccggcaccgacttcagtctgaatatacatcccatggaagaagacgataccgccatgtacttttgccaacaatctaaggaggtgccttggacgttcggcggcggtacgaagctggaaattaagggcggcgggggaagcggcggggggggatcaggcgggggtggctccggaggcggtggaagtatgggctggagttggatcttccttttccttctttctggtaccgcgggagtgcactctgaggtgcagctccagcagtccggccccgagctcgtcaagcctggggccagtgtcaagatttcctgtaaggcatctggatatacctttacagattacaatatgcattgggtgaaacagtcacatggaaagtcactcgagtggatcggatacatttacccttacaatggaggaaccggatataatcagaagtttaagagcaaggccacactcacggtggacaattcttcatctacagcctacatggatgttcggtctctgacttccgaggatagtgcggtgtattactgcgccaggggacgccccgctatggattactgggggcagggaacctctgtaacagttagctca

SEQ ID NO: 3 - DNA, 1044 bp, Artificial Sequence
  Synthetic single-chain variable fragment
atggccttaccagtgaccgccttgctcctgccgctggccttgctgctccacgccgccaggccggacttcgtgatgactcagtctcctagctccctgaccgtgacagccggcgagaaggtgaccatgtcctgcaaatctagtcagagtctgctgaactcaggcaatcagaagaactatctgacatggtacctgcagaagccagggcagccccctaaactgctgatctattgggccagcaccagggaatccggcgtgcccgacagattcaccggctccgggtctggaacagattttactctgaccatttcaagcgtgcaggccgaggacctggctgtgtactattgtcagaatgattacagctatccctacacatttggcgggggaactaagctggaaatcaaaggtggtggtggttctggtggtggtggttccggcggcggcggctccggtggtggtggatccgaggtgcagctgcagcagagtggacccgaactggtgaaacctggcgcctccgtgaaaatgtcttgcaaggctagtgggtacaccttcacagactactatatgaaatgggtgaagcagtcacacgggaagagcctggagtggatcggagatatcattccctctaacggcgccactttctacaatcagaagtttaaaggcaaggctactctgaccgtggaccggagctcctctaccgcctatatgcacctgaacagtctgacatcagaagatagcgctgtgtactattgtacacggtcccatctgctgagagcctcttggtttgcttattggggccaggggacactggtgactgtgagctccgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtg

SEQ ID NO: 4 - DNA, 252 bp, Homo sapiens
tccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatct

SEQ ID NO: 5 - DNA, 72 bp, Homo sapiens
ccattttttttctgctgcttcatcgctgtagccatgggaatccgtttcattattatggtaacaatatggagt

SEQ ID NO: 6 - DNA, 252 bp, Homo sapiens
cacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaa

SEQ ID NO: 7 - DNA, 73 bp, Homo sapiens
gtctctttctgcttggtgatggtactcctttttgcagtggacacaggactatatttctctgtgaagacaaaca

SEQ ID NO: 8 - DNA, 252 bp, Homo sapiens
tgtagaatctaccgcccttctgacaactctgtctctaagtccgtcagattctatctggtggtatctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagcccccattgcc

SEQ ID NO: 9 - DNA, 72 bp, Homo sapiens
ctggtgcctgtgttctgtggactcctcgtagccaagagcctggtgctgtcagccctgctcgtctggtggggg

SEQ ID NO: 10 - DNA, 252 bp, Homo sapiens
tccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggg

SEQ ID NO: 11 - DNA, 72 bp, Homo sapiens
gctggtacagtcctcctccttcgggctggattctatgctgtcagctttctctctgtggccgtgggcagcacc

SEQ ID NO: 12 - DNA, 252 bp, Homo sapiens
ttccccctgggccctgtgaccacagcccacagagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcgg

SEQ ID NO: 13 - DNA, 72 bp, Homo sapiens
atgggcctggcctttctagtcctggtggctctagtgtggttcctggttgaagactggctcagcaggaagagg

SEQ ID NO: 14 - DNA, 252 bp, Homo sapiens
agggaaggggaggcccatgaacgtaggctccctgcagtgcgcagcatcaacggaacattccaggccgactttcctctgggccctgccacccacggagggacctacagatgcttcggctctttccgtgacgctccctacgagtggtcaaactcgagtgatccactgcttgtttccgtcacaggaaacccttcaaatagttggccttcacccactgaaccaagctccaaaaccggtaaccccagacacctacat

SEQ ID NO: 15 - DNA, 69 bp, Homo sapiens
gttctgattgggacctcagtggtcaaaatccctttcaccatcctcctcttctttctccttcatcgctgg

SEQ ID NO: 16 - DNA, 210 bp, Homo sapiens
atgaataaacaaagaggaaccttctcagaagtgagtctggcccaggacccaaagcggcagcaaaggaaacctaaaggcaataaaagctccatttcaggaaccgaacaggaaatattccaagtagaattaaatcttcaaaatccttccctgaatcatcaagggattgataaaatatatgactgccaaggtttactgccacctccagagaag

SEQ ID NO: 17 - DNA, 78 bp, Homo sapiens
ctcactgccgaggtcctaggaatcatttgcattgtcctgatggccactgtgttaaaaacaatagttcttattcctttc

SEQ ID NO: 18 - DNA, 252 bp, Homo sapiens
gtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 19 - DNA, 72 bp, Homo sapiens
atctacatctgggcgcccttggccgggacttgtggggtccttctcctgtcactggttatcaccctttactgc

SEQ ID NO: 20 - DNA, 252 bp, Homo sapiens
gcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacacc

SEQ ID NO: 21 - DNA, 84 bp, Homo sapiens
attccgtggctcggccacctcctcgtgggcctcagcggggcttttggcttcatcatcttagtgtacttgctgatcaactgcagg

SEQ ID NO: 22 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 23 - DNA, 182 bp, Homo sapiens
aaggagaaggagagagagaagagatctatttacagagtcctgggatacacagaaggcacccaataactatagaagtcccatctctaccagtcaacctaccaatcaatccatggatgatacaagagaggatatttatgtcaactatccaaccttctctcgcagaccaaagactagagtttaag

SEQ ID NO: 24 - DNA, 512 bp, Homo sapiens
tttctcagggagtattgctaaaatgccaaaaaggaagttgttcaaatgccactcagtatgaggacactggagatctaaaagtgaataatggcacaagaagaaatataagtaataaggacctttgtgcttcgagatctgcagaccagacagtactatgccaatcagaatggctcaaataccaagggaagtgttattggttctctaatgagatgaaaagctggagtgacagttatgtgtattgtttggaaagaaaatctcatctactaatcatacatgaccaacttgaaatggcttttatacagaaaaacctaagacaattaaactacgtatggattgggcttaactttacctccttgaaaatgacatggacttgggtggatggttctccaatagattcaaagatattcttcataaagggaccagctaaagaaaacagctgtgctgccattaaggaaagcaaaattttctctgaaacctgcagcagtgttttcaaatggatttgtcagtattag

SEQ ID NO: 25 - DNA, 251 bp, Homo sapiens
agaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtg

SEQ ID NO: 26 - DNA, 242 bp, Homo sapiens
attccctatctttgtctactcagcgaacacagggccccgcagagtccgcaaggaacctagagtatgtttcagtgtctccaacgaacaacactgtgtatgcttcagtcactcattcaaacagggaaacagaaatctggacacctagagaaaatgatactatcacaatttactccacaattaatcattccaaagagagtaaacccactttttccagggcaactgcccttgacaatgtcgtgtaa

SEQ ID NO: 27 - DNA, 167 bp, Homo sapiens
agtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaag

SEQ ID NO: 28 - DNA, 351 bp, Homo sapiens
aaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaa

SEQ ID NO: 29 - DNA, 210 bp, Homo sapiens
atccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 30 - DNA, 144 bp, Homo sapiens
aagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatga

SEQ ID NO: 31 - DNA, 141 bp, Homo sapiens
aaggctctgatccacctgagcgacctccgggagtacaggcgctttgagaaggagaagctcaagtcccagtggaacaatgataatccccttttcaagagcgccaccacgacggtcatgaaccccaagtttgctgagagttag

SEQ ID NO: 32 - DNA, 144 bp, Homo sapiens
aaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaa

SEQ ID NO: 33 - DNA, 861 bp, Homo sapiens
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 34 - DNA, 573 bp, Homo sapiens
tataaagttgacttggttctgttctataggcgcatagcggaaagagacgagacactaacagatggtaaaacatatgatgcctttgtgtcttacctgaaagagtgtcatcctgagaataaagaagagtatacttttgctgtggagacgttacccagggtcctggagaaacagtttgggtataagttatgcatatttgaaagagatgtggtgcctggcggagctgttgtcgaggagatccattcactgatagagaaaagccggaggctaatcatcgttctcagccagagttacctgactaacggagccaggcgtgagctcgagagtggactccacgaagcactggtagagaggaagattaagatcatcttaattgagtttactccagccagcaacatcacctttctccccccgtcgctgaaactcctgaagtcctacagagttctaaaatggagggctgacagtccctccatgaactcaaggttctggaagaatcttgtttacctgatgcccgcaaaagccgtcaagccatggagagaggagtcggaggcgcggtctgttctctcagcaccttga

SEQ ID NO: 35 - DNA, 279 bp, Homo sapiens
aacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtga

SEQ ID NO: 36 - DNA, 660 bp, Homo sapiens
cattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 37 - DNA, 858 bp, Homo sapiens
agcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaa

SEQ ID NO: 38 - DNA, 1542 bp, Homo sapiens
cccctgagcatgcatcagcagcagcagctccagcaccagcagttccagaaggaactggagaagatccagctcctgcagcagcagcagcagcagctgcccttccacccacctggagacacggctcaggacggcgagctcctggacacgtctggcccgtactcagagagctcgggcaccagcagccccagcacgtcccccagggcctccaaccactcgctctgctccggcagctctgcctccaaggctggcagcagcccctccctggaacaagacgatggagatgaggaaaccagcgtggtgatagttgggaaaatttccttctgtcccaaggatgtcctgggccatggagctgagggcacaattgtgtaccggggcatgtttgacaaccgcgacgtggccgtgaagaggatcctccccgagtgttttagcttcgcagaccgtgaggtccagctgttgcgagaatcggatgagcacccgaacgtgatccgctacttctgcacggagaaggaccggcaattccagtacattgccatcgagctgtgtgcagccaccctgcaagagtatgtggagcagaaggactttgcgcatctcggcctggagcccatcaccttgctgcagcagaccacctcgggcctggcccacctccactccctcaacatcgttcacagagacctaaagccacacaacatcctcatatccatgcccaatgcacacggcaagatcaaggccatgatctccgactttggcctctgcaagaagctggcagtgggcagacacagtttcagccgccgatctggggtgcctggcacagaaggctggatcgctccagagatgctgagcgaagactgtaaggagaaccctacctacacggtggacatcttttctgcaggctgcgtcttttactacgtaatctctgagggcagccacccttttggcaagtccctgcagcggcaggccaacatcctcctgggtgcctgcagccttgactgcttgcacccagagaagcacgaagacgtcattgcacgtgaattgatagagaagatgattgcgatggatcctcagaaacgcccctcagcgaagcatgtgctcaaacacccgttcttctggagcctagagaagcagctccagttcttccaggacgtgagcgacagaatagaaaaggaatccctggatggcccgatcgtgaagcagttagagagaggcgggagagccgtggtgaagatggactggcgggagaacatcactgtccccctccagacagacctgcgtaaattcaggacctataaaggtggttctgtcagagatctcctccgagccatgagaaataagaagcaccactaccgggagctgcctgcagaggtgcgggagacgctggggtccctccccgacgacttcgtgtgctacttcacatctcgcttcccccacctcctcgcacacacctaccgggccatggagctgtgcagccacgagagactcttccagccctactacttccacgagcccccagagccccagcccccagtgactccagacgccctctga

(19) United States
(12) Patent Application Publication
(10) Pub. No.: US 2022/0073585 A1
(43) Pub. Date: Mar. 10, 2022

(54) CHIMERIC ANTIGEN RECEPTOR MEMORY-LIKE (CARML) NK CELLS AND METHODS OF MAKING AND USING SAME

(71) Applicant: Washington University, St. Louis, MO (US)

(72) Inventors: Todd A. Fehniger, St. Louis, MO (US); Melissa Berrien-Elliott, St. Louis, MO (US)

(21) Appl. No.: 17/291,959

(22) PCT Filed: Nov. 6, 2019
(86) PCT No.: PCT/US2019/060005

§ 371 Date: May 6, 2021

Related U.S. Application Data
(60) Provisional application No. 62/756,294, filed on Nov. 6, 2018.
(60) Provisional application No. 62/788,440, filed on Jan. 4, 2019.

(51) Int. Cl. C07K 14/705; C12N 5/0783; A61K 35/17; A61K 39/00

(52) U.S. Cl. CPC C07K 14/70517; C12N 5/0646; A61K 35/17; A61K 39/001112; C12N 2510/00; C07K 2317/622; C07K 2319/03; A61K 2039/5158; A61K 2039/5156; C07K 2317/53

(57) ABSTRACT

Among the various aspects of the present disclosure is the provision of a chimeric antigen receptor memory-like (CARML) NK cell and methods of making and using same.

### CROSS-REFERENCE TO RELATED APPLICATIONS

This application claims priority from U.S. Provisional Application Ser. No. 62/756,294 filed on 6 Nov. 2018 and 62/788,440 filed on 4 Jan. 2019, which are incorporated herein by reference in their entireties.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure generally relates to modified NK cells.

### SUMMARY OF THE INVENTION

Among the various aspects of the present disclosure is the provision of a memory-like chimeric antigen receptor (CARML) NK cells and methods of use thereof.

An aspect of the present disclosure provides for a chimeric antigen receptor (CAR) construct. In some embodiments, the construct comprises a targeting antibody fragment against a disease-associated antigen; a transmembrane domain; or at least one intracellular signaling domain. In some embodiments, the CAR construct is capable of being expressed or functioning in a memory-like natural killer (ML NK) cell.

In some embodiments, the disease-associated antigen is selected from the group consisting of CD19, CD33, CD123, CD20, BCMA, Mesothelin, EGFR, CD3, CD4 BAFF-R, EGFR, HER2, gp120, or gp41.

In some embodiments, the transmembrane domain is selected from the group consisting of NKG2D, FcγRIIIa, NKp44, NKp30, NKp46, actKIR, NKG2C, CD8a, or IL15Rb.

In some embodiments, the at least one intracellular signaling domain is selected from the group consisting of CD137/41BB, DNAM-1, NKp80, 2B4, NTBA, CRACC, CD2, CD27, one or more integrins, IL-15R, IL-18R, IL-12R, IL-21R, IRE1a, or combinations thereof.

In some embodiments, the at least one intracellular signaling domain is a transmembrane adapter.

In some embodiments, the CAR construct further comprises a transmembrane adapter or hinge.

In some embodiments, the transmembrane adapter is selected from the group consisting of FceR1γ, CD3ζ, DAP12, DAP10, or combinations thereof.

In some embodiments, the one or more integrins are selected from the group consisting of ITGB1, ITGB2, ITGB3, or combinations thereof.

In some embodiments, the targeting antibody fragment against a disease-associated antigen comprises a scFv selected from the group consisting of: (i) anti-CD19 scFv comprising an amino acid sequence of SEQ ID NO: 1; (ii) anti-CD33 scFv comprising an amino acid sequence of SEQ ID NO: 2; or (iii) anti-CD123 scFv comprising an amino acid sequence of SEQ ID NO: 3.

In some embodiments, the transmembrane domain is selected from the group consisting of: NKG2D comprising an amino acid sequence of SEQ ID NO: 5; FcγRIIIa comprising an amino acid sequence of SEQ ID NO: 7; NKp44 comprising an amino acid sequence of SEQ ID NO: 9; NKp30 comprising an amino acid sequence of SEQ ID NO: 11; NKp46 comprising an amino acid sequence of SEQ ID NO: 13; actKIR comprising an amino acid sequence of SEQ ID NO: 15; NKG2C comprising an amino acid sequence of SEQ ID NO: 17; CD8a comprising an amino acid sequence of SEQ ID NO: 19; or IL15Rb comprising an amino acid sequence of SEQ ID NO: 21.

In some embodiments, the hinge is selected from the group consisting of: NKG2D comprising an amino acid sequence of SEQ ID NO: 4; FcγRIIIa comprising an amino acid sequence of SEQ ID NO: 6; NKp44 comprising an amino acid sequence of SEQ ID NO: 8; NKp30 comprising an amino acid sequence of SEQ ID NO: 10; NKp46 comprising an amino acid sequence of SEQ ID NO: 12; actKIR comprising an amino acid sequence of SEQ ID NO: 14; NKG2C comprising an amino acid sequence of SEQ ID NO: 16; CD8a comprising an amino acid sequence of SEQ ID NO: 18; or IL15Rb comprising an amino acid sequence of SEQ ID NO: 20.

In some embodiments, the at least one intracellular signaling domain is selected from the group consisting of: CD137/41BB comprising an amino acid sequence of SEQ ID NO: 22; DNAM-1 comprising an amino acid sequence of SEQ ID NO: 23; NKp80 comprising an amino acid sequence of SEQ ID NO: 24; 2B4 comprising an amino acid sequence of SEQ ID NO: 25; NTBA comprising an amino acid sequence of SEQ ID NO: 26; CRACC comprising an amino acid sequence of SEQ ID NO: 27; CD2 comprising an amino acid sequence of SEQ ID NO: 28); CD27 comprising an amino acid sequence of SEQ ID NO: 29); integrins, ITGB1 comprising an amino acid sequence of SEQ ID NO: 30, ITGB2 comprising an amino acid sequence of SEQ ID NO: 31, or ITGB3 comprising an amino acid sequence of SEQ ID NO: 32; IL15RB comprising an amino acid sequence of SEQ ID NO: 33; IL18R comprising an amino acid sequence of SEQ ID NO: 34; IL12R, IL12RB1 comprising an amino acid sequence of SEQ ID NO: 35 or IL12RB2 comprising an amino acid sequence of SEQ ID NO: 36; IL21R comprising an amino acid sequence of SEQ ID NO: 37; IRE1a comprising an amino acid sequence of SEQ ID NO: 38; or combinations thereof.

Another aspect of the present disclosure provides for a memory-like natural killer (ML NK) cell comprising the CAR construct as described herein.

Another aspect of the present disclosure provides for a method of generating chimeric antigen receptor memory-like natural killer (CARML NK) cells. In some embodiments, the method comprises providing NK cells, activating cytokines comprising IL-12/15/18, or IL-15; contacting the NK cells or activating cytokines for an amount of time sufficient to form cytokine-activated memory-like (ML) NK cells; transducing a chimeric antigen receptor (CAR) via a viral vector into the cytokine-activated ML NK cells in the presence of IL-15 for an amount of time sufficient to virally transduce CAR into the cytokine-activated ML NK cells, resulting in CAR-transduced ML NK cells; or incubating the CAR-transduced ML NK cells in the presence of IL-15 for an amount of time sufficient to form CAR-expressing ML NK (CARML NK cells).

In some embodiments, the NK cells were isolated from peripheral blood mononuclear cells (PBMCs).

In some embodiments, the amount of time sufficient to form cytokine-activated NK cells is between about 8 and about 24 hours, about 12 hours, or about 16 hours.

In some embodiments, the amount of time sufficient to virally transduce CAR into the ML NK cells is between about 12 hours and about 24 hours.

In some embodiments, the amount of time sufficient to form ML NK cells expressing CAR (CARML NK cells) is at least between about 3 days and about 8 days or about 7 days.

In some embodiments, the viral vector comprises a chimeric antigen receptor (CAR) is a CAR lentivirus.

In some embodiments, the viral vector is a lentiviral vector selected from the group consisting of pMND-G, pMND-Lg, pMDN-REV, or combinations thereof.

In some embodiments, transducing a chimeric antigen receptor (CAR) via a viral vector into the cytokine-activated ML NK cells is performed in the absence of polybrene.

Another aspect of the present disclosure provides for a chimeric antigen receptor memory-like natural killer (CARML NK) cell made according to the method described herein comprising the CAR construct described herein.

Another aspect of the present disclosure provides for a method of inducing an immune response to a disease in a subject in need thereof. In some embodiments, the method comprises administering a chimeric antigen receptor memory like (CARML) NK cell to the subject, wherein the CARML NK cell comprises a chimeric antigen receptor (CAR) comprising a targeting antibody fragment against a disease-associated antigen; a transmembrane domain; or at least one intracellular signaling domain.

In some embodiments, the disease-associated antigen is selected from the group consisting of CD19, CD33, CD123, CD20, BCMA, Mesothelin, EGFR, CD3, CD4 BAFF-R, EGFR, HER2, gp120, or gp41.

In some embodiments, the transmembrane domain is selected from the group consisting of NKG2D, FcγRIIIa, NKp44, NKp30, NKp46, actKIR, NKG2C, CD8a, or IL15Rb.

In some embodiments, the at least one intracellular signaling domain is selected from the group consisting of CD137/41BB, DNAM-1, NKp80, 2B4, NTBA, CRACC, CD2, CD27, one or more integrins, IL-15R, IL-18R, IL-12R, IL-21R, IRE1a, or combinations thereof.

In some embodiments, the at least one intracellular signaling domain is a transmembrane adapter.

In some embodiments, the method further comprises a transmembrane adapter.

In some embodiments, the transmembrane adapter is selected from the group consisting of FceR1γ, CD3ζ, DAP12, DAP10, or combinations thereof.

In some embodiments, the one or more integrins are selected from the group consisting of ITGB1, ITGB2, ITGB3, or combinations thereof.

In some embodiments, the targeting antibody fragment against a disease-associated antigen comprises a scFv selected from the group consisting of: (i) anti-CD19 scFv comprising an amino acid sequence of SEQ ID NO: 1; (ii) anti-CD33 scFv comprising an amino acid sequence of SEQ ID NO: 2; or (iii) anti-CD123 scFv comprising an amino acid sequence of SEQ ID NO: 3.

In some embodiments, the transmembrane domain is selected from the group consisting of: NKG2D comprising an amino acid sequence of SEQ ID NO: 5; FcγRIIIa comprising an amino acid sequence of SEQ ID NO: 7; NKp44 comprising an amino acid sequence of SEQ ID NO: 9; NKp30 comprising an amino acid sequence of SEQ ID NO: 11; NKp46 comprising an amino acid sequence of SEQ ID NO: 13; actKIR comprising an amino acid sequence of SEQ ID NO: 15; NKG2C comprising an amino acid sequence of SEQ ID NO: 17; CD8a comprising an amino acid sequence of SEQ ID NO: 19; or IL15Rb comprising an amino acid sequence of SEQ ID NO: 21.

In some embodiments, the method further comprises a hinge selected from the group consisting of: NKG2D comprising an amino acid sequence of SEQ ID NO: 4; FcγRIIIa comprising an amino acid sequence of SEQ ID NO: 6; NKp44 comprising an amino acid sequence of SEQ ID NO: 8; NKp30 comprising an amino acid sequence of SEQ ID NO: 11; NKp46 comprising an amino acid sequence of SEQ ID NO: 12; actKIR comprising an amino acid sequence of SEQ ID NO: 14; NKG2C comprising an amino acid sequence of SEQ ID NO: 16; CD8α comprising an amino acid sequence of SEQ ID NO: 18; or IL15Rb comprising an amino acid sequence of SEQ ID NO: 20.

In some embodiments, the at least one intracellular signaling domain is selected from CD137/41BB comprising an amino acid sequence of SEQ ID NO: 22; DNAM-1 comprising an amino acid sequence of SEQ ID NO: 23; NKp80 comprising an amino acid sequence of SEQ ID NO: 24; 2B4 comprising an amino acid sequence of SEQ ID NO: 25; NTBA comprising an amino acid sequence of SEQ ID NO: 26; CRACC comprising an amino acid sequence of SEQ ID NO: 27; CD2 comprising an amino acid sequence of SEQ ID NO: 28); CD27 comprising an amino acid sequence of SEQ ID NO: 29); integrins, ITGB1 comprising an amino acid sequence of SEQ ID NO: 30, ITGB2 comprising an amino acid sequence of SEQ ID NO: 31, or ITGB3 comprising an amino acid sequence of SEQ ID NO: 32; IL15RB comprising an amino acid sequence of SEQ ID NO: 33; IL18R comprising an amino acid sequence of SEQ ID NO: 34; IL12R, IL12RB1 comprising an amino acid sequence of SEQ ID NO: 35 or IL12RB2 comprising an amino acid sequence of SEQ ID NO: 36; IL21R comprising an amino acid sequence of SEQ ID NO: 37; IRE1a comprising an amino acid sequence of SEQ ID NO: 38; or combinations thereof.

In some embodiments, the CAR construct is capable of expressing or functioning in a memory-like natural killer (ML NK) cell.

In some embodiments, the CARML NK cell induces an immune response to an antigen-specific target.

In some embodiments, the CARML NK cell reduces tumor burden.

In some embodiments, the targeting antibody fragment against a disease-associated antigen comprises a single chain variable fragment (scFv) against a disease-associated antigen.

In some embodiments, the subject has a disease having a disease-associated antigen.

In some embodiments, the antigen is a B cell antigen or the disease is selected from the group consisting of a hematological cancer, an autoimmune disease, or immune system disorders.

In some embodiments, the antigen is a tumor-associated antigen (TAA) or the disease is cancer.

In some embodiments, the CARML NK cell has an enhanced functional response against antigen targets or epitopes compared to a control.

In some embodiments, the control is an ML NK cell without CAR, an MLNK cell without a scFv, an NK cell with CAR, an NK cell with CAR scFv, an ML NK comprising a scFv not associated with a target, or NK comprising a scFv not associated with a target.

In some embodiments, the subject has cancer, an autoimmune condition, or an infectious disease (e.g., bacterial, viral).

Another aspect of the present disclosure provides for a method of administering CARML NK cells to a subject in need thereof. In some embodiments, the method comprises isolating NK cells from a subject or a donor; generating CARML NK cells according to the methods described herein; or administering a therapeutically effective amount of CARML NK cells into the subject.

In some embodiments, the therapeutically effective amount of CARML NK cells is about 10⁷ cell/kg.

In some embodiments, rhIL-2 or IL-15 is administered to the subject.

Another aspect of the present disclosure provides for a chimeric antigen receptor (CAR) construct comprising: (i) an anti-CD19 scFv comprising SEQ ID NO: 1, an anti-CD33 scFv comprising SEQ ID NO: 2, or an anti-CD123 scFv comprising SEQ ID NO: 3; (ii) a CD8α transmembrane domain comprising SEQ ID NO: 19, a NKp30 transmembrane domain comprising SEQ ID NO: 11, or a NKG2D transmembrane domain comprising SEQ ID NO: 5; and (iii) a CD137 intracellular signaling domain comprising SEQ ID NO: 22, a IL-15R intracellular signaling domain comprising SEQ ID NO: 33, or a2B4 intracellular signaling domain comprising SEQ ID NO: 25; wherein the CAR construct is capable of being expressed or functioning in a memory-like natural killer (ML NK) cell.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. Chimeric Antigen Receptor (CAR) Memory-Like (ML) NK cell generation. (A) CAR construct that targets CD19 antigen expressed on target cell. (B) Schema of retroviral transduction protocol. (C) NK cells transduced with CD19-CAR were rested for 5 days and GFP expression assessed by flow cytometry. Data are representative of 8 normal donors. Inset numbers represent the frequency of cells within the indicated gate.

FIG. 2. CARML NK cells exhibit more robust responses to antigen specific targets. CARML NK cells were generated as described in FIG. 1. Cells were stimulated with Rajis (CD19+ tumor cell line) for 6 hours and assessed by flow cytometry. (A) Schema of control GFP-only or GFP-CAR lentivirus. (B) Representative flow staining of NK cells transduced with control (GFP-only) or GFP-CAR lentivirus. Transduced cells are GFP+. (C) Representative IFN-γ and CD107a (degranulation staining from GFP-CAR GFP− and GFP-CAR GFP+ML NK cells. (D) Summary from C. Data are pooled from 2 normal donors.

FIG. 3. CARML NK cells exhibit more robust responses to antigen specific targets. CARML NK cells were generated as described in FIG. 1. Cells were stimulated with Rajis (CD19+ tumor cell line) for 6 hours and assessed by flow cytometry. Summary IFN-γ (A) and degranulation (CD107a, B) data demonstrating CARML NK cells respond robustly and specifically to CD19+ Raji targets. Data from 4 normal donors.

FIG. 4. Strategy for memory-like NK cell specific genetic modifications to optimize anti-tumor responses, cytokine production, cytotoxicity, proliferation, persistence.

FIG. 5. Strategy to tailor CARML NK to different functions, incorporating ML NK cell biology. (A) Intermediate activating CAR utilized in FIG. 1-FIG. 3 and FIG. 6-FIG. 8, with triggering via two well-studied signaling domains. (B) High level activation via multiple signaling mechanisms. (C) ML NK cell CAR engineered for moderate activation and enhanced NK persistence.

FIG. 6. CD19-CARML NK cells exhibit enhanced functional responses against Raji targets. Purified NK cells were activated with IL-12, IL-15, and IL-18 or control (IL-15) for 16 hours, washed, transduced with CD19-CAR lentivirus, and then rested in low concentrations of IL-15 to allow for CARML differentiation. (A) Schematic representation of the lentiviral cassette encoding the CD19-CAR. Transmembrane: CD8a, co-stimulatory domain: 4-1 BB, and stimulatory domain: CD3ζ, conjugated to a green fluorescent protein (GFP) marker via a P2A skip site. (B) Schema of memory-like (ML) NK cells in vitro experiments. (C) Our construct results in surface expression of CD19-CAR as determined by co expression of GFP and binding to soluble CD19 antigen. (D) Representative bivariate flow plots from memory-like NK cells transduced with CD19-CAR lentivirus showing IFNγ production and degranulation (CD107a expression) against CD19+ Raji targets on day 7 (gated on live CAR(GFP)− or CAR (GFP)+ NK cell populations). (E) Summary data showing enhanced IFNγ production and CD107a expression by CD19-CARML NK cells compared to both control ML NK cells and CD19-CAR NK cells (n=6; 3 independent experiments). Statistical analysis was done by two-tailed paired t-test; *p<0.05, **p<0.01, ***p<0.001.

FIG. 7. CD19-CARML NK cells display antigen (CD19) specific enhanced responses against tumor cell lines and primary follicular lymphoma targets. (A) In contrast to their enhanced degranulation and IFN-γ production against Raji target cells, CD19-CARML NK cells do not display enhanced responses against a CD19-negative Kasumi-3 cell line. (B) ML NK cells transduced with CD33-CD8a-41BB-CD3z-GFP CAR and stimulated with Raji targets did not have increased effector functions compared to controls (GFP−). (C) Representative flow cytometry data showing CD33/CD19-positive staining for each follicular lymphoma lymph node. (D-F) CD19-CARML exhibit significantly enhanced (D) IFN-γ, (E) degranulation, and (F) killing against primary CD19+ follicular lymphoma targets compared to ML NK cell controls and CD33 CARML. (G) Autologous CD19-CARML NK cells generated from lymphoma patient NK cells demonstrated significantly increased IFN-γ against their own respective CD19+ follicular lymphoma targets, compared to control ML NK cells (GFP−). Data represent two to three independent experiments and were compared using paired T-test.

FIG. 8. Studies demonstrate CD19-specific ML NK cells expand in vivo and control tumor burden in CD19+ Raji bearing mice. (A) Experimental design for (B) to (E). NOD-scid IL2Rγ^{null} (NSG) mice were challenged with 1×10⁶ luc-Raji cells. On day 4, mice were injected IV with human 19-CARML NK cells or human 33-CARML-NK cells as indicated. rhIL-15, recombinant human IL-15, QOD, every other day. (B) Representative flow cytometry at day 18 after transfer shows expansion of 19-CARML NK cells compared to CD33-CARML NK cells in the spleen. For each product, CAR expression was at about 20% prior to infusion. (C) In a separate experiment, mice were monitored for tumor burden using bioluminescence imaging (BLI) and survival (right).

FIG. 9. CD33-CARML NK cells display antigen (CD33) specific enhanced responses against tumor cell lines. (A) CD33-CARML Construct schema. Here the CD33-specific scFv was cloned in. (B) In contrast to their enhanced degranulation (CD107a) and IFN-γ production against CD33+ MOLM-13, CD33− CARML NK cells do not display enhanced responses against a CD33 negative Raji cell line. Data represent two to three independent experiments and were compared using paired T-test.

FIG. 10. CD123-CAR can be expressed in ML NK cells. The CD123-CAR construct is expressed in ML NK cells.

FIG. 11. CD19-CAR (IL2Rb)-ML NK cells display enhanced pSTAT-5 signaling in the presence of CD19+ Raji targets. (A) CD19-CAR (IL2Rb)-ML construct schema. Here the CD19-specific scFv was cloned in, but are utilizing the IL2Rb intracellular signaling domain. (B) The CD19 (IL2Rb) CAR construct can be expressed in ML NK cells. (C) Data demonstrating enhanced pSTAT-5 signaling is specific to CD19-CAR (IL2Rb) expressing ML NK cells compared to 19-CAR (41 BB/CD3z) expressing NK cells, which were both triggered with antigen-specific CD19⁺ Raji targets for 2 hours. MFI-Median fluorescence intensity.

FIG. 12. Generated constructs.

FIG. 13. Clinical processing for treating patients with (A) haplo/allogeneic CARML NK cells or (B) autologous CARML NK cells. Apheresis will be performed, NK cells will be purified and activated with IL-12/IL-15/IL-18 for about 12 hours. The NK cells will then be washed and spinfected with a CAR lentivirus, twice over about two days. The cells will be washed and infused into the patient at about 10⁷ cell/kg. In the haplo/allo setting the cells can be supported with rhIL-2 and in the autologous setting the cells can be supported with IL-15.

### DETAILED DESCRIPTION OF THE INVENTION

The present disclosure is based, at least in part, on the discovery that modifying the memory-like (ML) NK cells to incorporate a CAR suitable for use in a ML NK cell can result in the recognition of a broad array of targets (e.g., tumors, autoantibodies) and enhance their function, survival, or persistence via synthetic biology or genome editing. It is believed that the present disclosure is the first to design these CAR constructs capable of being incorporated into NK cells, more specifically, in ML NK cells. These CARML NK cell constructs can be used in the treatment of cancer (e.g., cancer immunotherapy) or immune related, or autoimmune diseases.

Described herein are chimeric antigen receptor (CAR) constructs capable of functioning or expressing in ML NK cells. ML NK cells have signaling molecules not present in other NK cells. Here is described the activation of NK cell and response to a target using CAR-modified ML NK cells. These CARML NK cells can target any antigen, for example an antigen associated with an infectious disease, a bacterial infection, a virus, a cancer, an autoimmune disease, or an immune disorder or dysfunction.

Described herein, the designed CARML NK cell constructs provide enhanced performance when compared to NK cells or ML NK cells without the CAR constructs.

Memory-Like Nk Cells

Natural Killer (NK) cells are cytotoxic innate lymphoid cells serving at the front line against infection and cancer. In inflammatory microenvironments, multiple soluble and contact-dependent signals modulate NK cell responsiveness. Besides their innate cytotoxic and immunostimulatory activity, it has been uncovered in recent years that NK cells constitute a heterogeneous and versatile cell subset. Persistent memory-like NK populations that mount a robust recall response have been reported during viral infection, contact hypersensitivity reactions, and after stimulation by pro-inflammatory cytokines or activating receptor pathways.

Here is described the generation, functionality, and clinical applicability of memory-like NK cells and the design of novel NK cell-based immunotherapies.

As described herein, the memory-like NK cell process has been improved using synthetic biology. Examples disclosed herein include a chimeric antibody receptor (CAR) specific for use in ML NK cells.

Memory-like NK cells are potent anti-leukemia effectors. A process was previously discovered to enhance NK cell anti-tumor responses, memory-like differentiation following combined cytokine receptor activation (cytokine-induced memory-like NK cells, CIML NK cells). This was advanced pre-clinically and then clinically in the setting of leukemia immunotherapy. The major inhibitory checkpoint on ML NK cells, NKG2A was also discovered (see e.g., as described in related U.S. patent application Ser. No. 15/983,275). Methods and compositions can be as described in related U.S. patent application Ser. No. 15/983,275 and is incorporated by reference in its entirety.

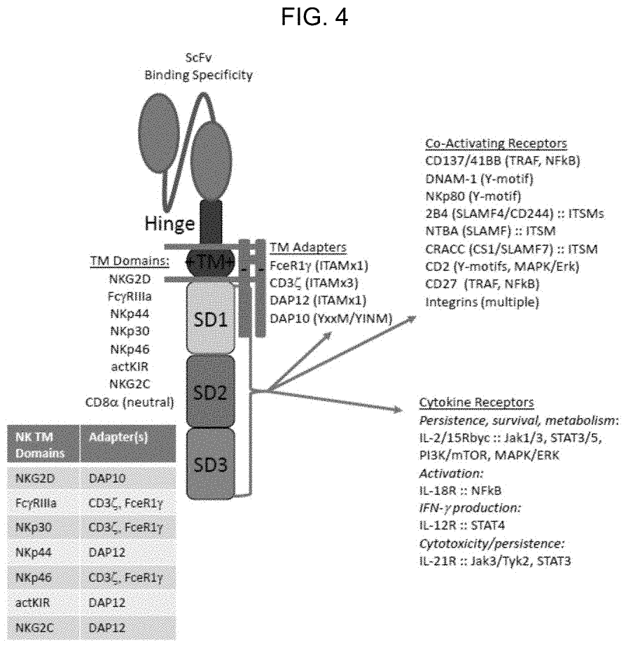

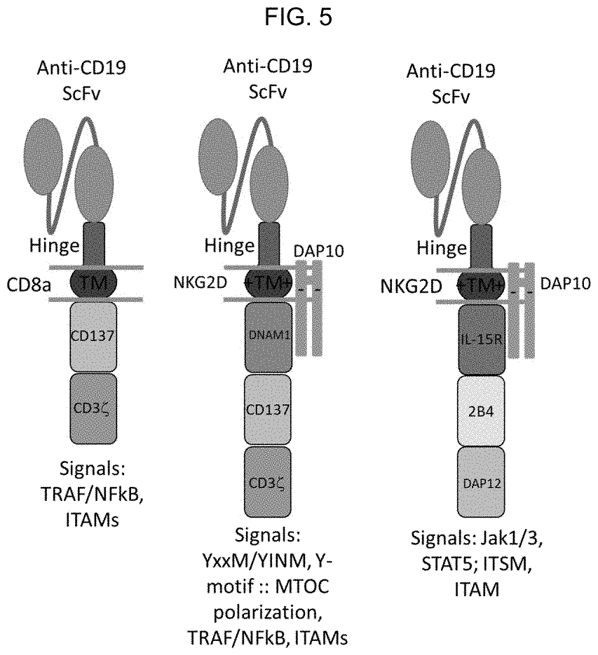

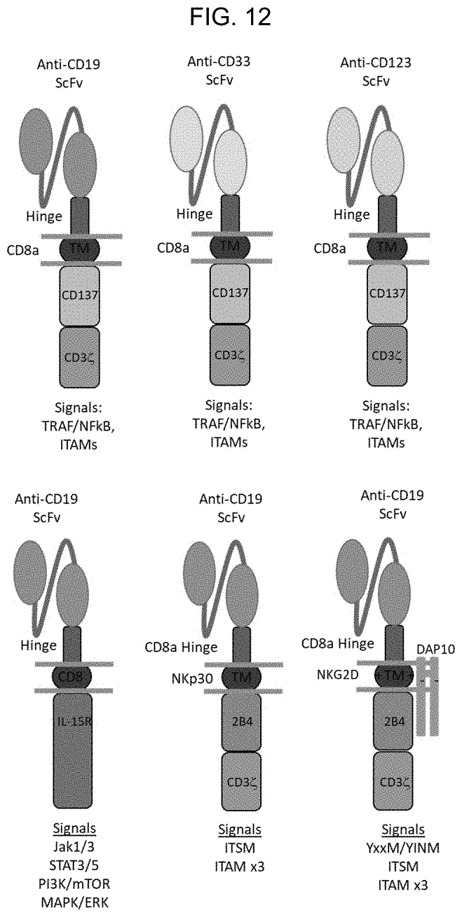

The present disclosure provides for a separate process improvement that provides new ways to have ML NK cells respond to recognize many antigens, for example, on a variety of tumor types, beyond the established biology of NK cell activating and inhibitory receptors. Specifically, this disclosure provides for the genetic modification of ML NK cells capable of responding, via a synthetic artificial receptor, using a chimeric antigen receptor (CAR). Here is described the use of a CD19, CD33, and CD123-recognizing receptors, directly, in ML NK cells against normally resistant B cell cancers with associated B cell antigens. This new platform can be used to perform many modifications of ML NK cells, to provide new recognition of antigens and tumors, provide new strategies to overcome inhibition, and enhance ML NK cell function, survival, and persistence. The design of these CARML NK cells provide novel possibilities and are based on ML NK cell biology (see e.g., FIG. 4, FIG. 5, FIG. 12).

Chimeric Antigen Receptor (CAR) Constructs

The present disclosure provides for ML NK cells modified with CARs. It is believed that the present disclosure is the first to design these CAR constructs capable of being incorporated into NK cells, more specifically, ML NK cells.

CAR designs are generally tailored to each cell type. The present disclosure is drawn to ML NK cells, but could be useful in other immune cell types. Disclosed herein are ML NK cells engineered to express chimeric antigen receptors (CARs).

CARs are designed in a modular fashion that comprise an extracellular target-binding domain, a hinge region, a transmembrane domain that anchors the CAR to the cell membrane, and one or more intracellular domains that transmit activation signals. Depending on the number of costimulatory domains, CARs can be classified into first (CD3z only), second (one costimulatory domain+CD3z), or third generation CARs (more than one costimulatory domain+CD3z). Introduction of CAR molecules into a ML NK cell successfully redirects the ML NK cell with additional antigen specificity and provides the necessary signals to drive full ML NK cell activation.

Because antigen recognition by CARML NK cells is based on the binding of the target-binding single-chain variable fragment (scFv) to intact surface antigens, targeting of tumor cells is not MHC restricted, co-receptor dependent, or dependent on processing and effective presentation of target epitopes.

Furthermore, the CAR construct moieties can be operably linked with a linker. A linker can be any nucleotide sequence capable of linking the moieties described herein. For example, the linker can be any amino acid sequence suitable for this purpose (e.g., of a length of 9 amino acids).

Targeting Antibody Fragment Against a Disease-Associated Antigen (e.g., Single-Chain Variable Fragments (scFvs))

Targeting antibody fragments against a disease-associated antigen can comprise Single-chain variable fragments (scFvs). scFvs, as described herein can be any scFv capable of binding to a target antigen or target antigen epitope. For example, the scFvs can target an antigen associated with an infectious disease, a bacterial infection, a virus, or a cancer. scFvs can be against any antigen known in the art, such as those described in U.S. application Ser. No. 15/179,472, and is incorporated by reference in its entirety.

Targeting antibody fragments or scFvs, as described herein, can be against any tumor-associated antigen (TAA). A TAA can be any antigen known in the art to be associated with tumors.

As described herein, examples of scFvs, CD19, CD33, and CD123 CARs were expressed on the ML NK cells. For example, CD19 can target cancer or deplete B cells for autoimmune diseases to remove autoantibodies. Other scFvs, such as scFvs that recognize: CD20, BCMA, Mesothelin, EGFR, CD3, CD4 BAFF-R, EGFR, HER2, HIV: gp120, or gp41 can also be incorporated into the CAR construct.

The antigen-binding capability of the CAR is defined by the extracellular scFv, not the targeted antigen. The format of a scFv is generally two variable domains linked by a flexible peptide sequence, either in the orientation VH-linker-VL or VL-linker-VH. The orientation of the variable domains within the scFv, depending on the structure of the scFv, may contribute to whether a CAR will be expressed on the ML NK cell surface or whether the CARML NK cells target the antigen and signal. In addition, the length and/or composition of the variable domain linker can contribute to the stability or affinity of the scFv.

scFvs are well known in the art to be used as a binding moiety in a variety of constructs (see e.g., Sentman 2014 Cancer J. 20 156-159; Guedan 2019 Mol Ther Methods Clin Dev. 12 145-156). Any scFv known in the art or generated against an antigen using means known in the art can be used as the binding moiety.

CAR scFv affinities, modified through mutagenesis of complementary-determining regions while holding the epitope constant, or through CAR development with scFvs derived from therapeutic antibodies against the same target, but not the same epitope, can change the strength of the ML NK cell signal and allow CARML NK cells to differentiate overexpressed antigens from normally expressed antigens. The scFv, a critical component of a CAR molecule, can be carefully designed and manipulated to influence specificity and differential targeting of tumors versus normal tissues. Given that these differences may only be measurable with CARML NK cells (as opposed to soluble antibodies), pre-clinical testing of normal tissues for expression of the target, and susceptibility to on-target toxicities, requires live-cell assays rather than immunohistochemistry on fixed tissues.

The scFvs described herein can be used for hematological malignancies such as AML, ALL, or Lymphoma, but can also be expanded for use in any malignancy, autoimmune, or infectious disease where a scFv can be generated against a target antigen or antigen epitope. For example, the constructs described herein can be used to treat or prevent autoimmunity associated with auto-antibodies (similar indications as rituximab for autoimmunity). As another example, the disclosed constructs can also be applied to virally infected cells, using scFv that can recognize viral antigens, for example gp120 and gp41 on HIV-infected cells.

scFv Sequences and Specificities:

Anti-CD19 scFv
(SEQ ID NO: 1)
ATGGCCCTGCCCGTGACCGCTCTCCTGCTGCCTCTGGCCCTGCTCCTCCATGCTGCCAG
ACCCGACATCCAGATGACACAGACAACCAGCAGCCTGTCCGCTTCCCTCGGAGACAGGG
TGACAATTTCCTGCAGGGCCAGCCAGGACATCAGCAAGTACCTGAACTGGTACCAGCAG
AAACCCGACGGCACCGTCAAGCTCCTGATCTACCACACCAGCAGACTGCACAGCGGAGT
GCCTTCCAGGTTCAGCGGCAGCGGCTCCGGCACCGATTACTCCCTGACCATTAGCAACT
TAGAACAGGAGGACATTGCCACCTACTTTTGTCAGCAGGGCAACACCCTCCCCTACACC
TTTGGAGGCGGAACCAAGTTAGAAATCACCGGCGGCGGCGGCAGCGGAGGAGGAGGCAG
CGGAGGCGGAGGCTCCGAGGTGAAACTGCAGGAGAGCGGCCCCGGACTGGTCGCCCCTA
GCCAATCCCTCTCCGTCACCTGCACCGTGAGCGGAGTGAGCCTGCCTGACTACGGAGTG
AGCTGGATCAGACAGCCCCCTAGGAAAGGACTGGAATGGCTGGGCGTGATTTGGGGCAG
CGAGACCACCTATTACAACAGCGCCCTGAAGTCCAGACTGACAATCATCAAGGACAATA
GCAAAAGCCAAGTGTTTCTGAAGATGAACAGCCTGCAGACCGATGACACCGCCATCTAT
TATTGCGCCAAGCACTACTACTACGGAGGAAGCTACGCTATGGATTATTGGGGCCAAGG
CACAAGCGTGACCGTCAGCAGCGCGGCCGCC
Anti-CD33 scFv
(SEQ ID NO: 2)
ATGGCCTTACCAGTGACCGCCTTGCTCCTGCCGCTGGCCTTGCTGCTCCACGCCGCCAG
GCCGATGGAAAAGGATACACTGTTGTTGTGGGTTCTGCTCCTGTGGGTGCCCGGCAGCA
CCGGAGATATTGTGCTGACGCAGTCTCCTGCATCACTCGCCGTGTCTCTGGGCCAGCGC
GCTACCATCAGCTGCAGAGCCTCTGAAAGTGTTGACAATTATGGAATTTCTTTCATGAA
TTGGTTCCAGCAGAAGCCTGGCCAGCCCCCGAAACTCCTCATATATGCCGCGTCTAATC
AGGGCTCTGGGGTCCCTGCTAGATTTTCTGGCAGCGGCTCCGGCACCGACTTCAGTCTG
AATATACATCCCATGGAAGAAGACGATACCGCCATGTACTTTTGCCAACAATCTAAGGA
GGTGCCTTGGACGTTCGGCGGCGGTACGAAGCTGGAAATTAAGGGCGGCGGGGGAAGCG
GCGGGGGGGGATCAGGCGGGGGTGGCTCCGGAGGCGGTGGAAGTATGGGCTGGAGTTGG
ATCTTCCTTTTCCTTCTTTCTGGTACCGCGGGAGTGCACTCTGAGGTGCAGCTCCAGCA
GTCCGGCCCCGAGCTCGTCAAGCCTGGGGCCAGTGTCAAGATTTCCTGTAAGGCATCTG
GATATACCTTTACAGATTACAATATGCATTGGGTGAAACAGTCACATGGAAAGTCACTC
GAGTGGATCGGATACATTTACCCTTACAATGGAGGAACCGGATATAATCAGAAGTTTAA
GAGCAAGGCCACACTCACGGTGGACAATTCTTCATCTACAGCCTACATGGATGTTCGGT
CTCTGACTTCCGAGGATAGTGCGGTGTATTACTGCGCCAGGGGACGCCCCGCTATGGAT
TACT GGGGGCAGGGAACCTCTGTAACAGTTAGCTCA
Anti-CD123 scFv
(SEQ ID NO: 3)
ATGGCCTTACCAGTGACCGCCTTGCTCCTGCCGCTGGCCTTGCTGCTCCACGCCGCCAG
GCCGGACTTCGTGATGACTCAGTCTCCTAGCTCCCTGACCGTGACAGCCGGCGAGAAGG
TGACCATGTCCTGCAAATCTAGTCAGAGTCTGCTGAACTCAGGCAATCAGAAGAACTAT
CTGACATGGTACCTGCAGAAGCCAGGGCAGCCCCCTAAACTGCTGATCTATTGGGCCAG
CACCAGGGAATCCGGCGTGCCCGACAGATTCACCGGCTCCGGGTCTGGAACAGATTTTA
CTCTGACCATTTCAAGCGTGCAGGCCGAGGACCTGGCTGTGTACTATTGTCAGAATGAT
TACAGCTATCCCTACACATTTGGCGGGGGAACTAAGCTGGAAATCAAAGGTGGTGGTGG
TTCTGGTGGTGGTGGTTCCGGCGGCGGCGGCTCCGGTGGTGGTGGATCCGAGGTGCAGC
TGCAGCAGAGTGGACCCGAACTGGTGAAACCTGGCGCCTCCGTGAAAATGTCTTGCAAG
GCTAGTGGGTACACCTTCACAGACTACTATATGAAATGGGTGAAGCAGTCACACGGGAA
GAGCCTGGAGTGGATCGGAGATATCATTCCCTCTAACGGCGCCACTTTCTACAATCAGA
AGTTTAAAGGCAAGGCTACTCTGACCGTGGACCGGAGCTCCTCTACCGCCTATATGCAC
CTGAACAGTCTGACATCAGAAGATAGCGCTGTGTACTATTGTACACGGTCCCATCTGCT
GAGAGCCTCTTGGTTTGCTTATTGGGGCCAGGGGACACTGGTGACTGTGAGCTCCGCTA
GCACCACGACGCCAGCGCCGCGACCACCAACACCGGCGCCCACCATCGCGTCGCAGCCC
CTGTCCCTGCGCCCAGAGGCGTGCCGGCCAGCGGCGGGGGGCGCAGTGCACACGAGGGG
GCTGGACTTCGCCTGTGATTTTTGGGTGCTGGTGGTGGTTGGTGGAGTCCTGGCTTGCT
ATAGCTTGCTAGTAACAGTGGCCTTTATTATTTTCTGGGTG

Transmembrane (TM) Domains and Adapters

The constructs described herein incorporate a transmembrane domain consisting of a hydrophobic a helix that spans the cell membrane. Although the main function of the transmembrane is to anchor the CAR in the ML NK cell membrane, previous evidence has also suggested that the transmembrane can be relevant for CAR cell function.

Others have previously looked at transmembrane (TM) domains for use in CAR, but do not work in known NK cells. The inventors discovered that, unexpectedly, the transmembrane domains that do not work in other NK cell CAR constructs work in ML NK cells, as described herein.

Here, it was shown that CD8 TM moiety was applicable for ML NK cells, because ML NK cells are more mature and have different characteristics than other NK cells. This TM domain does not work in other NK cells (see e.g., Li et al. 2018 Cell Stem Cell 23181-192).

The TM domain can be any TM domain suitable for use in an NK cell or ML NK cell. For example, the TM domain can be a sequence associated with NKG2D, FcγRIIIa, NKp44, NKp30, NKp46, actKIR, NKG2C, or CD8a.

NK cells express a number of transmembrane (TM) adapters that signal activation, that are triggered via association with activating receptors. This provides an NK cell specific signal enhancement via engineering the TM domains from activating receptors, and thereby harness endogenous adapters. The TM adapter can be any endogenous TM adapter capable of signaling activation. For example, the TM adapter can be FceR1γ (ITAMx1), CD3ζ (ITAMx3), DAP12 (ITAMx1), or DAP10 (YxxM/YINM).

It was discovered that ML NK cells have increased NKG2D, NKp30, and NKp44 expression, providing a rationale for their use in ML NK cells. As shown in FIG. 4, NK cells express a number of transmembrane (TM) adapters that signal activation, that are triggered via association with activating receptors. This provides an NK cell specific signal enhancement via engineering the TM domains from activating receptors, and thereby harness endogenous adapters.

Hinge (Spacer)

The hinge, also referred to as a spacer, is in the extracellular structural region of the CAR that separates the binding units from the transmembrane domain. The hinge can be any moiety capable of ensuring proximity of the CARML NK cell to the target (e.g., NKG2-based hinge, TMα-based hinge, CD8-based hinge). With the exception of a few CARs based on the entire extracellular moiety of a receptor, such as NKG2D, as described herein, the majority of CAR (such as CAR T) cells are designed with immunoglobulin (Ig)-like domain hinges.

Hinges generally supply stability for efficient CAR expression and activity. The NKG2 hinge (also in combination with the transmembrane domain), described herein also ensures proper proximity to target.

The hinge also provides flexibility to access the targeted antigen. The optimal spacer length of a given CAR can depend on the position of the targeted epitope. Long spacers can provide extra flexibility to the CAR and allow for better access to membrane-proximal epitopes or complex glycosylated antigens. CARs bearing short hinges can be more effective at binding membrane-distal epitopes. The length of the spacer can be important to provide adequate intercellular distance for immunological synapse formation. As such, hinges may be optimized for individual epitopes accordingly.

Here, the hinge is operably linked to the transmembrane domain.

Hinge/Transmembrane (TM) Domain Sequences

NKG2D (Hinge (SEQ ID NO: 4)/ TM (SEQ ID NO: 5))
TCCACAAGAATCAAGATCTTCCCTCTCTGAGCAGGAATCCTTTGTGCATTGAAGACTTT
AGATTCCTCTCTGCGGTAGACGTGCACTTATAAGTATTTGATGGGGTGGATTCGTGGTC
GGAGGTCTCGACACAGCTGGGAGATGAGTGAATTTCATAATTATAACTTGGATCTGAAG
AAGAGTGATTTTTCAACACGATGGCAAAAGCAAAGATGTCCAGTAGTCAAAAGCAAATG
TAGAGAAAATGCATCT/CCATTTTTTTTCTGCTGCTTCATCGCTGTAGCCATGGGAATC
CGTTTCATTATTATGGTAACAATATGGAGT
FcyRIlla (Hinge (SEQ ID NO: 6)/ TM (SEQ ID NO: 7))
CACCTGAGGTGTCACAGCTGGAAGAACACTGCTCTGCATAAGGTCACATATTTACAGAA
TGGCAAAGGCAGGAAGTATTTTCATCATAATTCTGACTTCTACATTCCAAAAGCCACAC
TCAAAGACAGCGGCTCCTACTTCTGCAGGGGGCTTTTTGGGAGTAAAAATGTGTCTTCA
GAGACTGTGAACATCACCATCACTCAAGGTTTGGCAGTGTCAACCATCTCATCATTCTT
TCCACCTGGGTACCAA/GTCTCTTTCTGCTTGGTGATGGTACTCCTTTTTGCAGTGGAC
ACAGGACTATATTTCTCTGTGAAGACAAACA
NKp44 (Hinge (SEQ ID NO: 8)/ TM (SEQ ID NO: 9))
TGTAGAATCTACCGCCCTTCTGACAACTCTGTCTCTAAGTCCGTCAGATTCTATCTGGT
GGTATCTCCAGCCTCTGCCTCCACACAGACCTCCTGGACTCCCCGCGACCTGGTCTCTT
CACAGACCCAGACCCAGAGCTGTGTGCCTCCCACTGCAGGAGCCAGACAAGCCCCTGAG
TCTCCATCTACCATCCCTGTCCCTTCACAGCCACAGAACTCCACGCTCCGCCCTGGCCC
TGCAGCCCCCATTGCC/CTGGTGCCTGTGTTCTGTGGACTCCTCGTAGCCAAGAGCCTG
GTGCTGTCAGCCCTGCTCGTCTGGTGGGGG
NKp30 (Hinge (SEQ ID NO: 10)/ TM (SEQ ID NO: 11))
TCCGTCACGTGGTTCCGAGATGAGGTGGTTCCAGGGAAGGAGGTGAGGAATGGAACCCC
AGAGTTCAGGGGCCGCCTGGCCCCACTTGCTTCTTCCCGTTTCCTCCATGACCACCAGG
CTGAGCTGCACATCCGGGACGTGCGAGGCCATGACGCCAGCATCTACGTGTGCAGAGTG
GAGGTGCTGGGCCTTGGTGTCGGGACAGGGAATGGGACTCGGCTGGTGGTGGAGAAAGA
ACATCCTCAGCTAGGG/GCTGGTACAGTCCTCCTCCTTCGGGCTGGATTCTATGCTGTC
AGCTTTCTCTCTGTGGCCGTGGGCAGCACC
NKp46 (Hinge (SEQ ID NO: 12)/ TM (SEQ ID NO: 13))
TTCCCCCTGGGCCCTGTGACCACAGCCCACAGAGGGACATACCGATGTTTTGGCTCCTA
TAACAACCATGCCTGGTCTTTCCCCAGTGAGCCAGTGAAGCTCCTGGTCACAGGCGACA
TTGAGAACACCAGCCTTGCACCTGAAGACCCCACCTTTCCTGCAGACACTTGGGGCACC
TACCTTTTAACCACAGAGACGGGACTCCAGAAAGACCATGCCCTCTGGGATCACACTGC
CCAGAATCTCCTTCGG/ATGGGCCTGGCCTTTCTAGTCCTGGTGGCTCTAGTGTGGTTC
CTGGTTGAAGACTGGCTCAGCAGGAAGAGG
Activating KIR (KIR2DS4) (Hinge (SEQ ID NO: 14)/ TM (SEQ ID NO: 15))
AGGGAAGGGGAGGCCCATGAACGTAGGCTCCCTGCAGTGCGCAGCATCAACGGAACATT
CCAGGCCGACTTTCCTCTGGGCCCTGCCACCCACGGAGGGACCTACAGATGCTTCGGCT
CTTTCCGTGACGCTCCCTACGAGTGGTCAAACTCGAGTGATCCACTGCTTGTTTCCGTC
ACAGGAAACCCTTCAAATAGTTGGCCTTCACCCACTGAACCAAGCTCCAAAACCGGTAA
CCCCAGACACCTACAT/GTTCTGATTGGGACCTCAGTGGTCAAAATCCCTTTCACCATC
CTCCTCTTCTTTCTCCTTCATCGCTGG
NKG2C (Hinge (SEQ ID NO: 16)/ TM (SEQ ID NO: 17))
ATGAATAAACAAAGAGGAACCTTCTCAGAAGTGAGTCTGGCCCAGGACCCAAAGCGGCA
GCAAAGGAAACCTAAAGGCAATAAAAGCTCCATTTCAGGAACCGAACAGGAAATATTCC
AAGTAGAATTAAATCTTCAAAATCCTTCCCTGAATCATCAAGGGATTGATAAAATATAT
GACTGCCAAGGTTTACTGCCACCTCCAGAGAAG/CTCACTGCCGAGGTCCTAGGAATCA
TTTGCATTGTCCTGATGGCCACTGTGTTAAAAACAATAGTTCTTATTCCTTTC
CD8a (Hinge (SEQ ID NO: 18)/ TM (SEQ ID NO: 19))
GTCCTCACCCTGAGCGACTTCCGCCGAGAGAACGAGGGCTACTATTTCTGCTCGGCCCT
GAGCAACTCCATCATGTACTTCAGCCACTTCGTGCCGGTCTTCCTGCCAGCGAAGCCCA
CCACGACGCCAGCGCCGCGACCACCAACACCGGCGCCCACCATCGCGTCGCAGCCCCTG
TCCCTGCGCCCAGAGGCGTGCCGGCCAGCGGCGGGGGGCGCAGTGCACACGAGGGGGCT
GGACTTCGCCTGTGAT/ATCTACATCTGGGCGCCCTTGGCCGGGACTTGTGGGGTCCTT
CTCCTGTCACTGGTTATCACCCTTTACTGC
IL15Rb (Hinge (SEQ ID NO: 20)/ TM (SEQ ID NO: 21))
GCCTCCCACTACTTTGAAAGACACCTGGAGTTCGAGGCCCGGACGCTGTCCCCAGGCCA
CACCTGGGAGGAGGCCCCCCTGCTGACTCTCAAGCAGAAGCAGGAATGGATCTGCCTGG
AGACGCTCACCCCAGACACCCAGTATGAGTTTCAGGTGCGGGTCAAGCCTCTGCAAGGC
GAGTTCACGACCTGGAGCCCCTGGAGCCAGCCCCTGGCCTTCAGGACAAAGCCTGCAGC
CCTTGGGAAGGACACC/ATTCCGTGGCTCGGCCACCTCCTCGTGGGCCTCAGCGGGGCT
TTTGGCTTCATCATCTTAGTGTACTTGCTGATCAACTGCAGG

Intracellular Signaling Domain (Costimulatory Domains)

The present disclosure provides for an intracellular signaling domain useful in ML NK cells. For example, others using NK cells were not able to use CD137 (4-1BB) in the NK cells, but surprisingly, these and others can work in the ML NK cells. The CAR construct can comprise one or more intracellular signaling domains.

NK cells can also utilize co-activating receptors to amplify activating signals. Signaling domains/motifs (SD) may be harnessed that are selectively expressed in ML NK cells (e.g., DNAM-1, CD137, CD2). Importantly, NK cells receive homeostasis, proliferation, and persistence signals from cytokine receptors, most notably the IL-2/15R. CARML NK cells may be further tailored to result in certain outcomes, including cytokine production, cytotoxicity, and long-term persistence.

In some embodiments, an intracellular signaling domain can be any co-activating receptor capable of functioning in an NK cell (e.g., a ML NK cell). For example, a co-activating receptor can be CD137/41BB (TRAF, NFkB), DNAM-1 (Y-motif), NKp80 (Y-motif), 2B4 (SLAMF)::ITSM, CRACC (CS1/SLAMF7)::ITSM, CD2 (Y-motifs, MAPK/Erk), CD27 (TRAF, NFkB), or integrins (e.g., multiple integrins).

In some embodiments, an intracellular signaling domain can be a cytokine receptor capable of functioning in an NK cell (e.g., a ML NK cell). For example, a cytokine receptor can be a cytokine receptor associated with persistence, survival, or metabolism, such as IL-2/15Rbyc::Jak1/3, STAT3/5, PI3K/mTOR, MAPK/ERK. As another example, a cytokine receptor can be a cytokine receptor associated with activation, such as IL-18R::NFkB. As another example, a cytokine receptor can be a cytokine receptor associated with IFN-γ production, such as IL-12R STAT4. As another example, a cytokine receptor can be a cytokine receptor associated with cytotoxicity or persistence, such as IL-21R::Jak3/Tyk2, or STAT3.

As another example, an intracellular signaling domain can be a TM adapter, such as FceR1γ (ITAMx1), CD3 (ITAMx3), DAP12 (ITAMx1), or DAP10 (YxxM/YINM).

As another example, CAR intracellular signaling domains (also known as endodomains) can be derived from costimulatory molecules from the CD28 family (such as CD28 and ICOS) or the tumor necrosis factor receptor (TNFR) family of genes (such as 4-1BB, OX40, or CD27). The TNFR family members signal through recruitment of TRAF proteins and are associated with cellular activation, differentiation and survival.

As another example, CD28 and 4-1BB have been widely used costimulatory endodomains in CARs in T cells, but it is believed this is the first time these endodomains have been shown to work in NK cells. Clinical trials with CARs incorporating CD28 or 4-1 BB intracellular domains showed similar response rates in patients with hematologic malignancies for T cells, but has yet to be shown in NK cells until now.

The high effector function and self-limited expansion of CD28-based CARs may be ideal to transiently treat diseases with a rapid tumor elimination and short-term persistence of the CAR in ML NK cells (i.e., as a bridge therapy for allogeneic hematopoietic stem cell transplantation). Furthermore, 4-1BB-based CARs may be used to treat diseases in which complete response may require sustained NK cell persistence.

Other domains, such as incorporation of ICOS can be incorporated into a CARML NK cell. Recent data suggest that various lymphocyte subsets require distinct costimulation signals for optimal function and persistence. The ICOS intracellular domain can enhance the persistence of CARML NK cells and the 4-1BB intracellular domain can provide optimal persistence in CARML NK cells.

In some embodiments, the CARML NK cell can join the properties of different intracellular domains in one single ML NK cell by combining two or more intracellular domains in a CAR. For example, such combinations can include one intracellular domain from the CD28 family and one intracellular domain from the TNFR family, resulting in the simultaneous activation of different signaling pathways.

Each costimulatory domain can have unique properties. Differences in the affinity of the scFv, the intensity of antigen expression, the probability of off-tumor toxicity, or the disease to be treated may influence the selection of the intracellular domain.

Intracellular Signaling Domain Sequences

CD137/41BB
(SEQ ID NO: 22)
aaacggggcagaaagaaactcctgtatatattcaaacaaccatttatgagaccagtaca
aactactcaagaggaagatggctgtagctgccgatttccagaagaagaagaaggaggat
gtgaactg
DNAM-1
(SEQ ID NO: 23)
aaggagaaggagagagagaagagatctatttacagagtcctgggatacacagaaggcac
ccaataactatagaagtcccatctctaccagtcaacctaccaatcaatccatggatgat
acaagagaggatatttatgtcaactatccaaccttctctcgcagaccaaagactagagt
ttaag
NKp80
(SEQ ID NO: 24)
tttctcagggagtattgctaaaatgccaaaaaggaagttgttcaaatgccactcagtat
gaggacactggagatctaaaagtgaataatggcacaagaagaaatataagtaataagga
cctttgtgcttcgagatctgcagaccagacagtactatgccaatcagaatggctcaaat
accaagggaagtgttattggttctctaatgagatgaaaagctggagtgacagttatgtg
tattgtttggaaagaaaatctcatctactaatcatacatgaccaacttgaaatggcttt
tatacagaaaaacctaagacaattaaactacgtatggattgggcttaactttacctcct
tgaaaatgacatggacttgggtggatggttctccaatagattcaaagatattcttcata
aagggaccagctaaagaaaacagctgtgctgccattaaggaaagcaaaattttctctga
aacctgcagcagtgttttcaaatggatttgtcagtattag
2B4
(SEQ ID NO: 25)
agaccagtcccaaggaatttttgacaatttacgaagatgtcaaggatctgaaaaccagg
agaaatcacgagcaggagcagacttttcctggaggggggagcaccatctactctatgat
ccagtcccagtcttctgctcccacgtcacaagaacctgcatatacattatattcattaa
ttcagccttccaggaagtctggatccaggaagaggaaccacagcccttccttcaatagc
actatctatgaagtg
NTBA
(SEQ ID NO: 26)
attccctatctttgtctactcagcgaacacagggccccgcagagtccgcaaggaaccta
gagtatgtttcagtgtctccaacgaacaacactgtgtatgcttcagtcactcattcaaa
cagggaaacagaaatctggacacctagagaaaatgatactatcacaatttactccacaa
ttaatcattccaaagagagtaaacccactttttccagggcaactgcccttgacaatgtc
gtgtaa
CRACC
(SEQ ID NO: 27)
agtacattgaagagaagaagagagtggacatttgtcgggaaactcctaacatatgcccc
cattctggagagaacacagagtacgacacaatccctcacactaatagaacaatcctaaa
ggaagatccagcaaatacggtttactccactgtggaaataccgaaaaag
CD2
(SEQ ID NO: 28)
aaaaggaaaaaacagaggagtcggagaaatgatgaggagctggagacaagagcccacag
agtagctactgaagaaaggggccggaagccccaccaaattccagcttcaacccctcaga
atccagcaacttcccaacatcctcctccaccacctggtcatcgttcccaggcacctagt
catcgtcccccgcctcctggacaccgtgttcagcaccagcctcagaagaggcctcctgc
tccgtcgggcacacaagttcaccagcagaaaggcccgcccctccccagacctcgagttc
agccaaaacctccccatggggcagcagaaaactcattgtccccttcctctaattaa
CD27
(SEQ ID NO: 29)
atccttgtgatcttctctggaatgttccttgttttcaccctggccggggccctgttcct
ccatcaacgaaggaaatatagatcaaacaaaggagaaagtcctgtggagcctgcagagc
cttgtcgttacagctgccccagggaggaggagggcagcaccatccccatccaggaggat
taccgaaaaccggagcctgcctgctccccctga

Integrins

A. ITGB1
(SEQ ID NO: 30)
aagcttttaatgataattcatgacagaagggagtttgctaaatttgaaaaggagaaaat
gaatgccaaatgggacacgggtgaaaatcctatttataagagtgccgtaacaactgtgg
tcaatccgaagtatgagggaaaatga
B. ITGB2
(SEQ ID NO: 31)
aaggctctgatccacctgagcgacctccgggagtacaggcgctttgagaaggagaagct
caagtcccagtggaacaatgataatccccttttcaagagcgccaccacgacggtcatga
accccaagtttgctgagagttag
C. ITGB3
(SEQ ID NO: 32)
aaactcctcatcaccatccacgaccgaaaagaattcgctaaatttgaggaagaacgcgc
cagagcaaaatgggacacagccaacaacccactgtataaagaggccacgtctaccttca
ccaatatcacgtaccggggcacttaa
IL15RB
(SEQ ID NO: 33)
aactgcaggaacaccgggccatggctgaagaaggtcctgaagtgtaacaccccagaccc
ctcgaagttcttttcccagctgagctcagagcatggaggagacgtccagaagtggctct
cttcgcccttcccctcatcgtccttcagccctggcggcctggcacctgagatctcgcca
ctagaagtgctggagagggacaaggtgacgcagctgctcctgcagcaggacaaggtgcc
tgagcccgcatccttaagcagcaaccactcgctgaccagctgcttcaccaaccagggtt
acttcttcttccacctcccggatgccttggagatagaggcctgccaggtgtactttact
tacgacccctactcagaggaagaccctgatgagggtgtggccggggcacccacagggtc
ttccccccaacccctgcagcctctgtcaggggaggacgacgcctactgcaccttcccct
ccagggatgacctgctgctcttctcccccagtctcctcggtggccccagccccccaagc
actgcccctgggggcagtggggccggtgaagagaggatgcccccttctttgcaagaaag
agtccccagagactgggacccccagcccctggggcctcccaccccaggagtcccagacc
tggtggattttcagccaccccctgagctggtgctgcgagaggctggggaggaggtccct
gacgctggccccagggagggagtcagtttcccctggtccaggcctcctgggcaggggga
gttcagggcccttaatgctcgcctgcccctgaacactgatgcctacttgtccctccaag
aactccagggtcaggacccaactcacttggtgtag
IL18R
(SEQ ID NO: 34)
tataaagttgacttggttctgttctataggcgcatagcggaaagagacgagacactaac
agatggtaaaacatatgatgcctttgtgtcttacctgaaagagtgtcatcctgagaata
aagaagagtatacttttgctgtggagacgttacccagggtcctggagaaacagtttggg
tataagttatgcatatttgaaagagatgtggtgcctggcggagctgttgtcgaggagat
ccattcactgatagagaaaagccggaggctaatcatcgttctcagccagagttacctga
ctaacggagccaggcgtgagctcgagagtggactccacgaagcactggtagagaggaag
attaagatcatcttaattgagtttactccagccagcaacatcacctttctccccccgtc
gctgaaactcctgaagtcctacagagttctaaaatggagggctgacagtccctccatga
actcaaggttctggaagaatcttgtttacctgatgcccgcaaaagccgtcaagccatgg
agagaggagtcggaggcgcggtctgttctctcagcaccttga

IL12R

IL12RB1
(SEQ ID NO: 35)
aacagggccgcacggcacctgtgcccgccgctgcccacaccctgtgccagctccgccat
tgagttccctggagggaaggagacttggcagtggatcaacccagtggacttccaggaag
aggcatccctgcaggaggccctggtggtagagatgtcctgggacaaaggcgagaggact
gagcctctcgagaagacagagctacctgagggtgcccctgagctggccctggatacaga
gttgtccttggaggatggagacaggtgcaaggccaagatgtga
IL12RB2
(SEQ ID NO: 36)
cattacttccagcaaaaggtgtttgttctcctagcagccctcagacctcagtggtgtag
cagagaaattccagatccagcaaatagcacttgcgctaagaaatatcccattgcagagg
agaagacacagctgcccttggacaggctcctgatagactggcccacgcctgaagatcct
gaaccgctggtcatcagtgaagtccttcatcaagtgaccccagttttcagacatccccc
ctgctccaactggccacaaagggaaaaaggaatccaaggtcatcaggcctctgagaaag
acatgatgcacagtgcctcaagcccaccacctccaagagctctccaagctgagagcaga
caactggtggatctgtacaaggtgctggagagcaggggctccgacccaaagcccgaaaa
cccagcctgtccctggacggtgctcccagcaggtgaccttcccacccatgatggctact
taccctccaacatagatgacctcccctcacatgaggcacctctcgctgactctctggaa
gaactggagcctcagcacatctccctttctgttttcccctcaagttctcttcacccact
caccttctcctgtggtgataagctgactctggatcagttaaagatgaggtgtgactccc
tcatgctctga
IL21R
(SEQ ID NO: 37)
agcctgaagacccatccattgtggaggctatggaagaagatatgggccgtccccagccc
tgagcggttcttcatgcccctgtacaagggctgcagcggagacttcaagaaatgggtgg
gtgcacccttcactggctccagcctggagctgggaccctggagcccagaggtgccctcc
accctggaggtgtacagctgccacccaccacggagcccggccaagaggctgcagctcac
ggagctacaagaaccagcagagctggtggagtctgacggtgtgcccaagcccagcttct
ggccgacagcccagaactcggggggctcagcttacagtgaggagagggatcggccatac
ggcctggtgtccattgacacagtgactgtgctagatgcagaggggccatgcacctggcc
ctgcagctgtgaggatgacggctacccagccctggacctggatgctggcctggagccca
gcccaggcctagaggacccactcttggatgcagggaccacagtcctgtcctgtggctgt
gtctcagctggcagccctgggctaggagggcccctgggaagcctcctggacagactaaa
gccaccccttgcagatggggaggactgggctgggggactgccctggggtggccggtcac
ctggaggggtctcagagagtgaggcgggctcacccctggccggcctggatatggacacg
tttgacagtggctttgtgggctctgactgcagcagccctgtggagtgtgacttcaccag
ccccggggacgaaggacccccccggagctacctccgccagtgggtggtcattcctccgc
cactttcgagccctggaccccaggccagctaa
IRE1a
(SEQ ID NO: 38)
cccctgagcatgcatcagcagcagcagctccagcaccagcagttccagaaggaactgga
gaagatccagctcctgcagcagcagcagcagcagctgcccttccacccacctggagaca
cggctcaggacggcgagctcctggacacgtctggcccgtactcagagagctcgggcacc
agcagccccagcacgtcccccagggcctccaaccactcgctctgctccggcagctctgc
ctccaaggctggcagcagcccctccctggaacaagacgatggagatgaggaaaccagcg
tggtgatagttgggaaaatttccttctgtcccaaggatgtcctgggccatggagctgag
ggcacaattgtgtaccggggcatgtttgacaaccgcgacgtggccgtgaagaggatcct
ccccgagtgttttagcttcgcagaccgtgaggtccagctgttgcgagaatcggatgagc
acccgaacgtgatccgctacttctgcacggagaaggaccggcaattccagtacattgcc
atcgagctgtgtgcagccaccctgcaagagtatgtggagcagaaggactttgcgcatct
cggcctggagcccatcaccttgctgcagcagaccacctcgggcctggcccacctccact
ccctcaacatcgttcacagagacctaaagccacacaacatcctcatatccatgcccaat
gcacacggcaagatcaaggccatgatctccgactttggcctctgcaagaagctggcagt
gggcagacacagtttcagccgccgatctggggtgcctggcacagaaggctggatcgctc
cagagatgctgagcgaagactgtaaggagaaccctacctacacggtggacatcttttct
gcaggctgcgtcttttactacgtaatctctgagggcagccacccttttggcaagtccct
gcagcggcaggccaacatcctcctgggtgcctgcagccttgactgcttgcacccagaga
agcacgaagacgtcattgcacgtgaattgatagagaagatgattgcgatggatcctcag
aaacgcccctcagcgaagcatgtgctcaaacacccgttcttctggagcctagagaagca
gctccagttcttccaggacgtgagcgacagaatagaaaaggaatccctggatggcccga
tcgtgaagcagttagagagaggcgggagagccgtggtgaagatggactggcgggagaac
atcactgtccccctccagacagacctgcgtaaattcaggacctataaaggtggttctgt
cagagatctcctccgagccatgagaaataagaagcaccactaccgggagctgcctgcag
aggtgcgggagacgctggggtccctccccgacgacttcgtgtgctacttcacatctcgc
ttcccccacctcctcgcacacacctaccgggccatggagctgtgcagccacgagagact
cttccagccctactacttccacgagcccccagagccccagcccccagtgactccagacg
ccctctga

Extracellular Signaling Domain

Optionally, an extracellular signaling domain can be incorporated into the CAR construct to propagate signaling. The extracellular signaling domain can be cloned into the hinge region, such as a CD8 hinge, but can be chosen based on the target.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

Described herein is a method of generating chimeric antigen receptor memory-like natural killer (CARML NK) cells. The isolated NK cells can be activated using cytokines, such as IL-12/15/18. The NK cells can be incubated in the presence of the cytokines for an amount of time sufficient to form cytokine-activated memory-like (ML) NK cells. For example, the amount of time sufficient to form cytokine-activated memory-like (ML) NK cells can be between about 8 and about 24 hours, about 12 hours, or about 16 hours. As another example, the amount of time sufficient to form cytokine-activated memory-like (ML) NK cells can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

Next, the chimeric antigen receptor (CAR) can be transduced via a viral vector (e.g., lentivirus) into the cytokine-activated ML NK cells in the presence of IL-15 for an amount of time sufficient to virally transduce CAR into the cytokine-activated ML NK cells, resulting in CAR-transduced ML NK cells. For example, the amount of time sufficient to form CAR-transduced ML NK cells can be between about 12 hours and about 24 hours. As another example, the amount of time sufficient to virally transduce CAR into the ML NK cells (forming CAR-transduced ML NK cells) can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

Next, the CAR-transduced ML NK cells can be incubated in the presence of IL-15 for an amount of time sufficient to express the vector and to form CAR-expressing ML NK (CARML NK cells). For example, the amount of time sufficient to form CARML NK cells can be between about 3 days and about 8 days. As an example, the amount of time sufficient to form CARML NK cells can be at least about 1 day; about 2 days; about 3 days; about 4 days; about 5 days; about 6 days; about 7 days; about 8 days; about 9 days; about 10 days; about 11 days; about 12 days; about 13 days; or about 14 days.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, intranasal, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating a proliferative disease, disorder, or condition, infectious disease, or immune disorder in a subject in need administration of a therapeutically effective amount of NK cell-based therapy (e.g., using genetically modified NK cells). The disclosed NK-cell based therapy can be used as a treatment for cancer (e.g., as an immunotherapy drug), for an autoimmune disease (e.g., treatment to deplete B cells), or for an infectious disease.

The scFvs described herein can be used for hematological malignancies such as AML, ALL, or Lymphoma, but can also be expanded for use in any malignancy, autoimmune, or infectious disease where a scFv can be generated against a target. For example, the constructs described herein can be used to treat or prevent autoimmunity associated with auto-antibodies (similar indications as rituximab for autoimmunity). As another example, the disclosed constructs can also be applied to virally infected cells, using a scFv that can recognize viral antigens, for example gp120 and gp41 on HIV-infected cells.

Methods described herein are generally performed on a subject in need thereof. A subject in need of the therapeutic methods described herein can be a subject having, diagnosed with, suspected of having, or at risk for developing a proliferative disease, disorder, or condition; an immune disorder; or an infectious disease. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of a NK cell-based treatment is, for example, that amount that would cause the desired therapeutic effect in a subject while minimizing undesired side effects. In various embodiments, an effective amount of a NK cell-based treatment described herein can substantially inhibit a disease, disorder, or condition, slow the progress of a disease, disorder, or condition, or limit the development of a disease, disorder, or condition.

Substantially can be any large portion up to totality. Thus “substantially blocked or inhibited”, or “substantially removed” can be nearly or nearly completely blocked, inhibited, or removed.

According to the methods described herein, administration can be parenteral, pulmonary, oral, topical, intradermal, intramuscular, intraperitoneal, intravenous, subcutaneous, intranasal, epidural, ophthalmic, buccal, or rectal administration. Preferably, NK cells can be administered as an intravenous infusion.

When used in the treatments described herein, a therapeutically effective amount of a NK cell-based treatment can be employed in a purified form or, where such forms exist, in pharmaceutically acceptable form and with or without a pharmaceutically acceptable excipient. For example, the compounds of the present disclosure can be administered, at a reasonable benefit/risk ratio applicable to any medical treatment, in a sufficient amount to inhibit a disease, disorder, or condition, slow the progress of a disease, disorder, or condition, or limit the development of a disease, disorder, or condition.

The amount of NK cell-based treatment (e.g., CARML NK cells) described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the NK cell-based treatment can occur as a single event or over a time course of treatment. For example, NK cell-based treatment can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a disease, disorder, or condition, such as chemotherapy, immunotherapy, or checkpoint blockade therapy. For example, a subject can be administered at least one therapeutic agent selected from an interferon; a checkpoint inhibitor antibody; an antibody-drug conjugate (ADC); an anti-HLA-DR antibody; or an anti-CD74 antibody. Other examples can include a therapeutic agent selected from a second antibody or antigen-binding fragment thereof, a drug, a toxin, an enzyme, a cytotoxic agent, an anti-angiogenic agent, a pro-apoptotic agent, an antibiotic, a hormone, an immunomodulator, a cytokine, a chemokine, an antisense oligonucleotide, a small interfering RNA (siRNA), a boron compound, or a radioisotope.

A NK cell-based treatment can be administered simultaneously or sequentially with another agent, such as an antibiotic, an anti-inflammatory, or another agent. For example, a NK cell-based treatment can be administered simultaneously with another agent, such as an antibiotic or an anti-inflammatory. Simultaneous administration can occur through administration of separate compositions, each containing one or more of a NK cell-based treatment, an antibiotic, an anti-inflammatory, or another agent. Simultaneous administration can occur through administration of one composition containing two or more of a NK cell-based treatment, an antibiotic, an anti-inflammatory, or another agent. A NK cell-based treatment can be administered sequentially with an antibiotic, an anti-inflammatory, or another agent. For example, a NK cell-based treatment can be administered before or after administration of an antibiotic, an anti-inflammatory, or another agent.

Methods and compositions as described herein can be used for the prevention, treatment, or slowing the progression of cancer, autoimmune conditions associated with autoantibodies, immune disorder, or infectious diseases (e.g., bacterial, viral). The disclosed CARML NK cell constructs can be designed to incorporate a targeting antibody fragment against a disease-associated antigen, such as scFvs that target cancer or an infectious disease. As described herein, targeting antibody fragments against a disease-associated antigens are well known.

For example, the cancer can a hematological cancer or a cancer with a solid tumor. For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

As another example, the autoimmune condition or immune disorder can be Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Bab disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum, Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inverse); Hypogammalglobulinemia; IgA Nephropathy; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus, Ligneous conjunctivitis; Linear IgA disease (LAD); Lupus; Lyme disease chronic; Meniere's disease; Microscopic polyangiitis (MPA); Mixed connective tissue disease (MCTD), Mooren's ulcer; Mucha-Habermann disease; Multifocal Motor Neuropathy (MMN) or MMNCB, Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (POD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, III; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA), Pyoderma gangrenosum, Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC), Undifferentiated connective tissue disease (UCTD), Uveitis; Vasculitis; Vitiligo; or Vogt-Koyanagi-Harada Disease.

As another example the autoimmune condition or immune disorder can be an autoinflammatory disease. The autoinflammatory can be Familial Mediterranean Fever (FMF), neonatal Onset Multisystem Inflammatory Disease (NOMID), Tumor Necrosis Factor Receptor-Associated Periodic Syndrome (TRAPS), Deficiency of the Interleukin-1 Receptor Antagonist (DIRA), Behcet's Disease, or Chronic Atypical Neutrophilic Dermatosis with Lipodystrophy and Elevated Temperature (CANDLE).

As another example, the treatment of an infectious disease can be any bacterial infection or viral infection, using a scFv that can recognize antigens, such as antigens on HIV infected cells. The infectious disease can be Acute Flaccid Myelitis (AFM); Anaplasmosis; Anthrax; Babesiosis; Botulism; Brucellosis; Campylobacteriosis; Carbapenem-resistant Infection (CRE/CRPA); Chancroid; Chikungunya Virus Infection (Chikungunya); Chlamydia; Ciguatera (Harmful Algae Blooms (HABs)); Clostridium Difficile Infection; Clostridium Perfringens (Epsilon Toxin); Coccidioidomycosis fungal infection (Valley fever); Creutzfeldt-Jacob Disease, transmissible spongiform encephalopathy (CJD); Cryptosporidiosis (Crypto); Cyclosporiasis; Dengue, 1,2,3,4 (Dengue Fever); Diphtheria; E. coli infection, Shiga toxin-producing (STEC); Eastern Equine Encephalitis (EEE); Ebola Hemorrhagic Fever (Ebola); Ehrlichiosis; Encephalitis, Arboviral or parainfectious; Enterovirus Infection, Non-Polio (Non-Polio Enterovirus); Enterovirus Infection, D68 (EV-D68); Giardiasis (Giardia); Glanders; Gonococcal Infection (Gonorrhea); Granuloma inguinale; Haemophilus Influenza disease, Type B (Hib or H-flu); Hantavirus Pulmonary Syndrome (HPS); Hemolytic Uremic Syndrome (HUS); Hepatitis A (Hep A); Hepatitis B (Hep B); Hepatitis C (Hep C); Hepatitis D (Hep D); Hepatitis E (Hep E); Herpes; Herpes Zoster, zoster VZV (Shingles); Histoplasmosis infection (Histoplasmosis); Human Immunodeficiency Virus/AIDS (HIV/AIDS); Human Papillomavirus (HPV), Influenza (Flu); Legionellosis (Legionnaires Disease); Leprosy (Hansens Disease); Leptospirosis; Listeriosis (Listeria); Lyme Disease; Lymphogranuloma venereum infection (LGV); Malaria; Measles; Melioidosis; Meningitis, Viral (Meningitis, viral); Meningococcal Disease, Bacterial (Meningitis, bacterial); Middle East Respiratory Syndrome Coronavirus (MERS-CoV); Mumps; Norovirus; Paralytic Shellfish Poisoning (Paralytic Shellfish Poisoning, Ciguatera); Pediculosis (Lice, Head and Body Lice); Pelvic Inflammatory Disease (PID); Pertussis (Whooping Cough); Plague; Bubonic, Septicemic, Pneumonic (Plague); Pneumococcal Disease (Pneumonia); Poliomyelitis (Polio); Powassan; Psittacosis (Parrot Fever); Pthiriasis (Crabs; Pubic Lice Infestation); Pustular Rash diseases (Small pox, monkeypox, cowpox); Q-Fever; Rabies; Ricin Poisoning; Rickettsiosis (Rocky Mountain Spotted Fever); Rubella, Including congenital (German Measles); Salmonellosis gastroenteritis (Salmonella); Scabies Infestation (Scabies); Scombroid; Septic Shock (Sepsis); Severe Acute Respiratory Syndrome (SARS); Shigellosis gastroenteritis (Shigella); Smallpox; Staphyloccal Infection, Methicillin-resistant (MRSA); Staphylococcal Food Poisoning, Enterotoxin—B Poisoning (Staph Food Poisoning); Staphylococcal Infection, Vancomycin Intermediate (VISA); Staphylococcal Infection, Vancomycin Resistant (VRSA); Streptococcal Disease, Group A (invasive) (Strep A (invasive)); Streptococcal Disease, Group B (Strep-B); Streptococcal Toxic-Shock Syndrome, STSS, Toxic Shock (STSS, TSS); Syphilis, primary, secondary, early latent, late latent, congenital; Tetanus Infection, tetani (Lock Jaw); Trichomoniasis (Trichomonas infection); Trichonosis Infection (Trichinosis); Tuberculosis (TB); Tuberculosis (Latent) (LTBI); Tularemia (Rabbit fever); Typhoid Fever, Group ID, Typhus; Vaginosis, bacterial (Yeast Infection); Vaping-Associated Lung Injury (e-Cigarette Associated Lung Injury); Varicella (Chickenpox); Vibrio cholerae (Cholera); Vibriosis (Vibrio); Viral Hemorrhagic Fever (Ebola, Lassa, Marburg); West Nile Virus; Yellow Fever; Yersenia (Yersinia); or Zika Virus Infection (Zika).

Administration

An aspect of the present disclosure provides for NK cells (e.g., CARML NK cells, modified NK cells, pre-activated NK cells, NKG2A-blocked NK cells, pre-activated and NKG2A-blocked NK cells) to be directly administered to a subject.

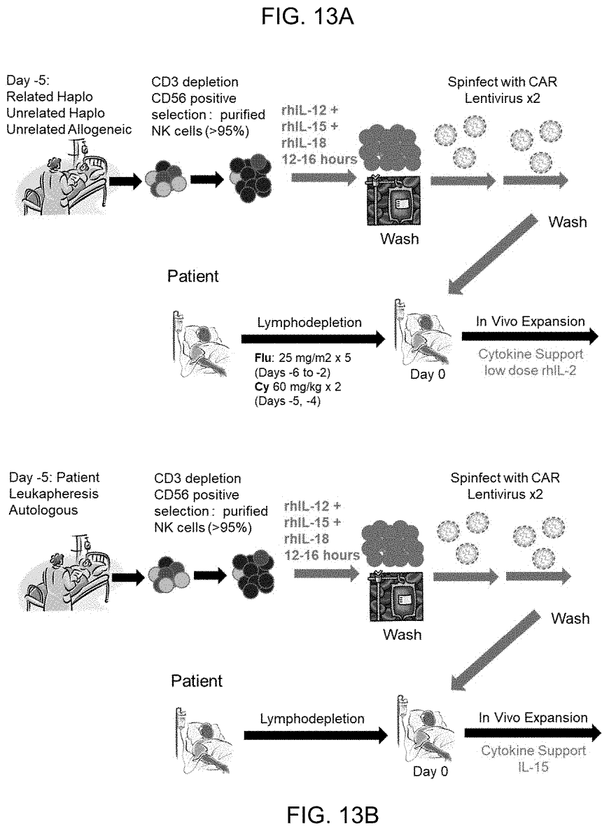

As described herein (see e.g., Example 2), clinical processing and treating patients with haplo/allogeneic CARML NK cells or autologous CARML NK cells (see e.g., FIG. 13) can be performed using the CARML NK cells as described herein.

Apheresis (e.g., the removal of blood plasma from the body by the withdrawal of blood, its separation into plasma and cells, and the reintroduction of the cells) can be performed on the subject.

The NK cells can be purified and activated with IL-12/IL-15/IL-18 for about 12 hours. The NK cells can be washed and spinfected with CAR lentivirus (e.g., twice over about two days). The cells can be washed and infused into the patient at about 10⁷ cell/kg. In the haplo/allo setting the cells can be supported with rhIL-2 and in the autologous setting the cells can be supported with IL-15.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Memory-Like Chimeric Antigen Receptor (CARML) NK Cell

The following example describes CARML NK cells, methods of generating CARML NK cells, and characterization of the CARML NK cells.

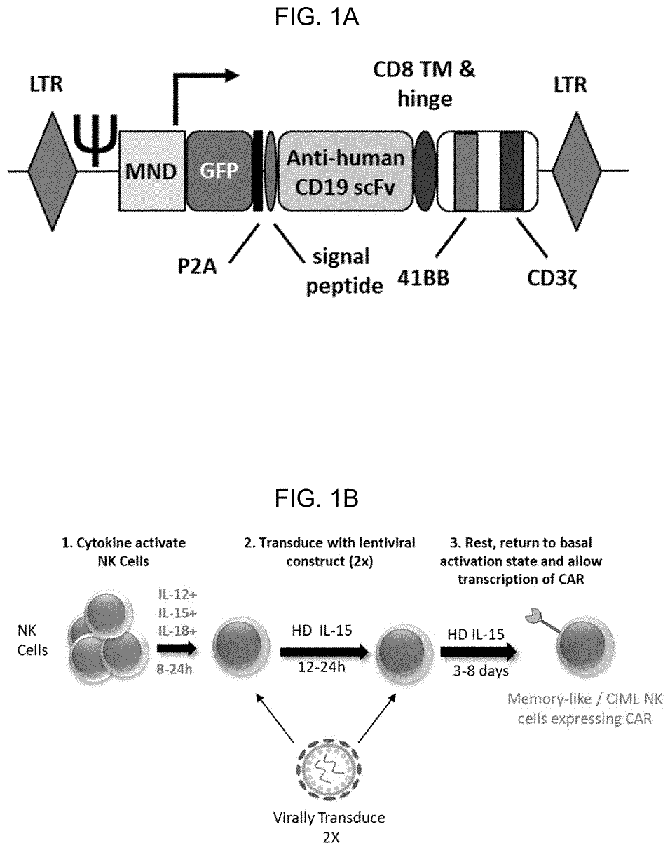

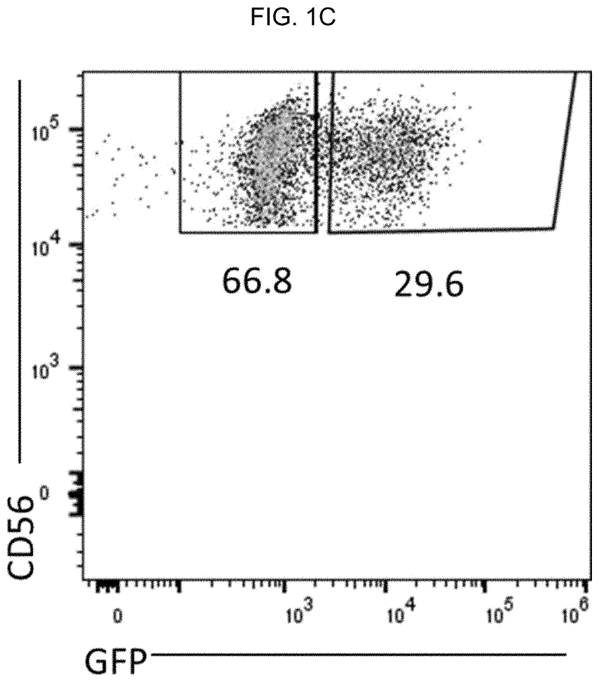

It was shown that CARML NK cells with a CAR construct that targets CD19, CD22, and CD123 antigens expressed on target cell can be generated (see e.g., FIG. 1, FIG. 12).

CARML NK Cells Exhibit More Robust Immune Responses to Antigen-Specific Targets

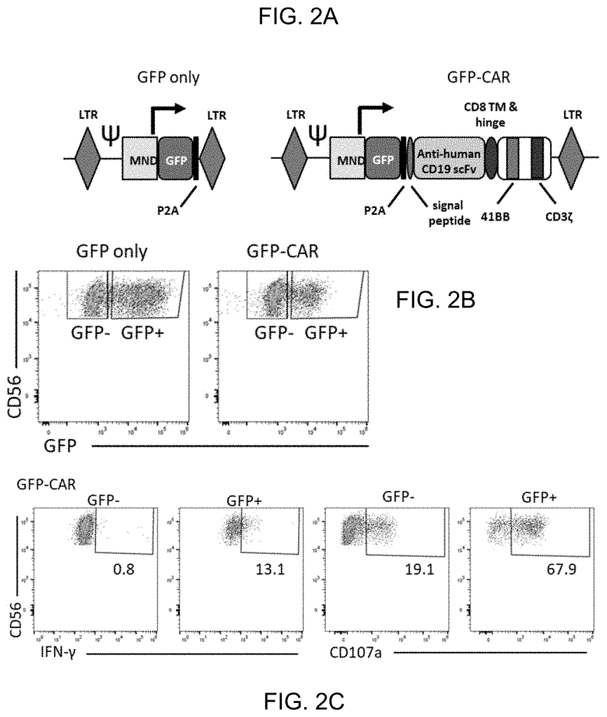

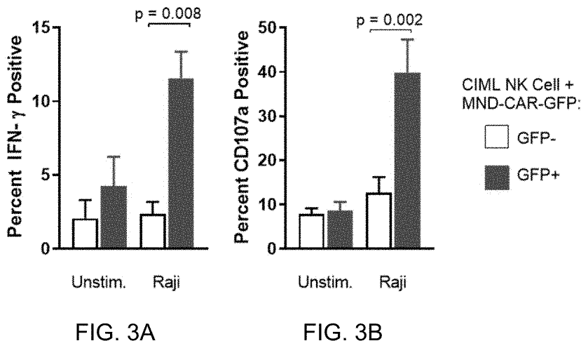

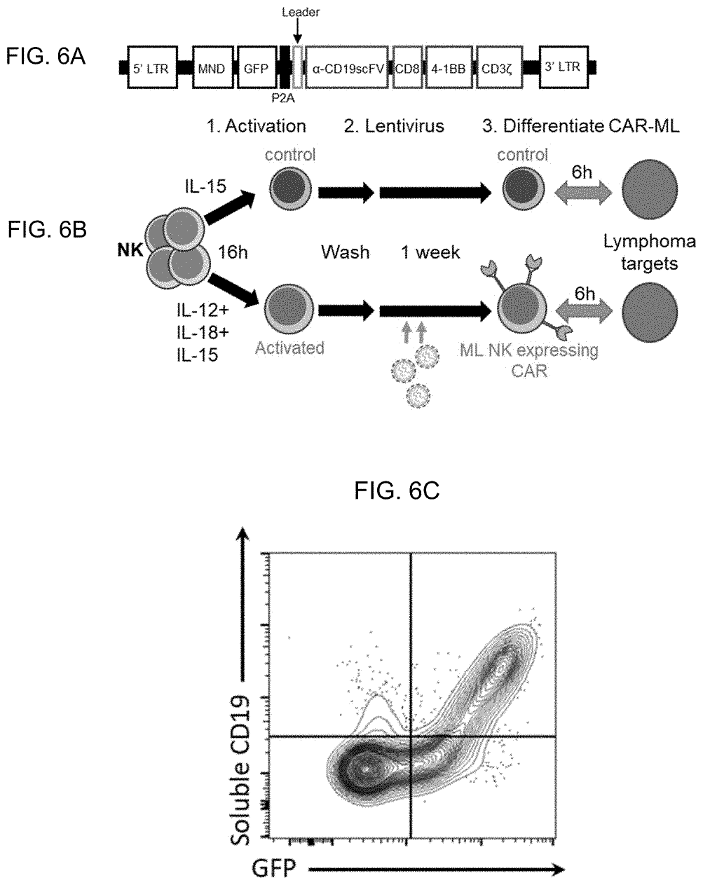

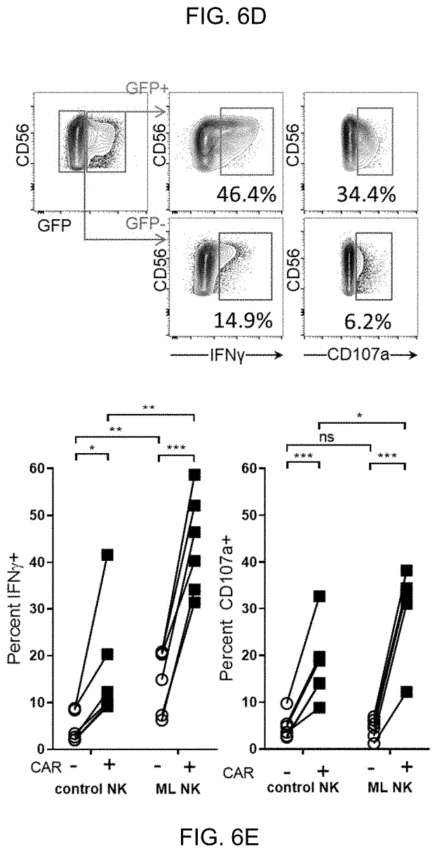

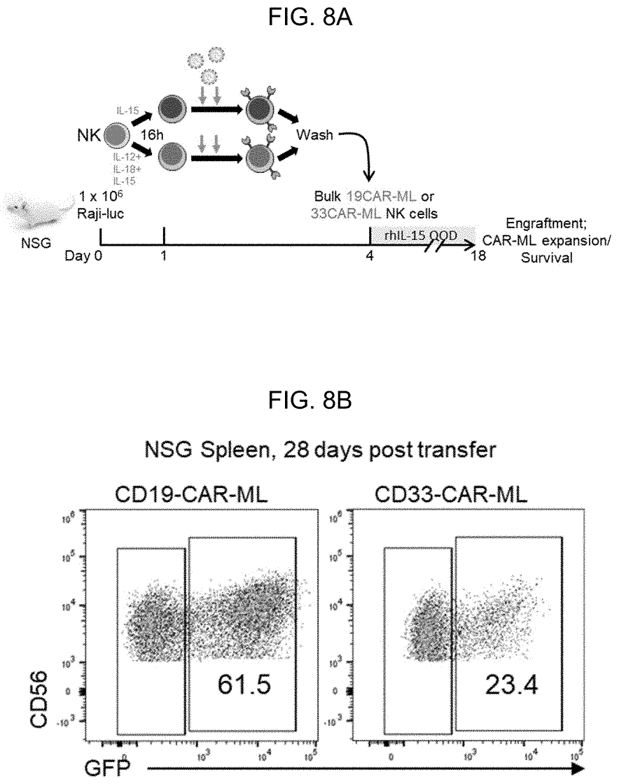

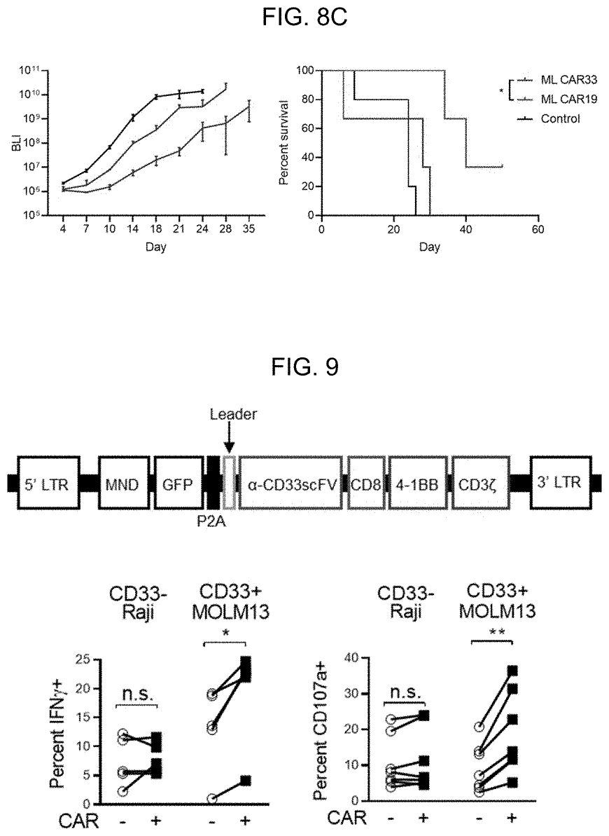

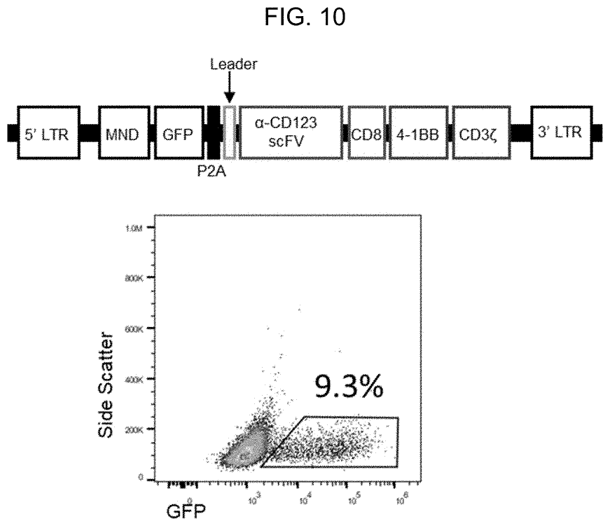

It was shown that CARML NK cells exhibit more robust responses to antigen specific targets (see e.g., FIG. 2, FIG. 3, FIG. 6, FIG. 9). CARML NK cells were generated as described in FIG. 1, FIG. 6, and FIG. 8.

Cells were stimulated with Rajis (CD19+ tumor cell line) for 6 hours and assessed by flow cytometry. Data demonstrated CARML NK cells respond robustly and specifically to CD19+ Raji targets (see e.g., FIG. 3, FIG. 6).

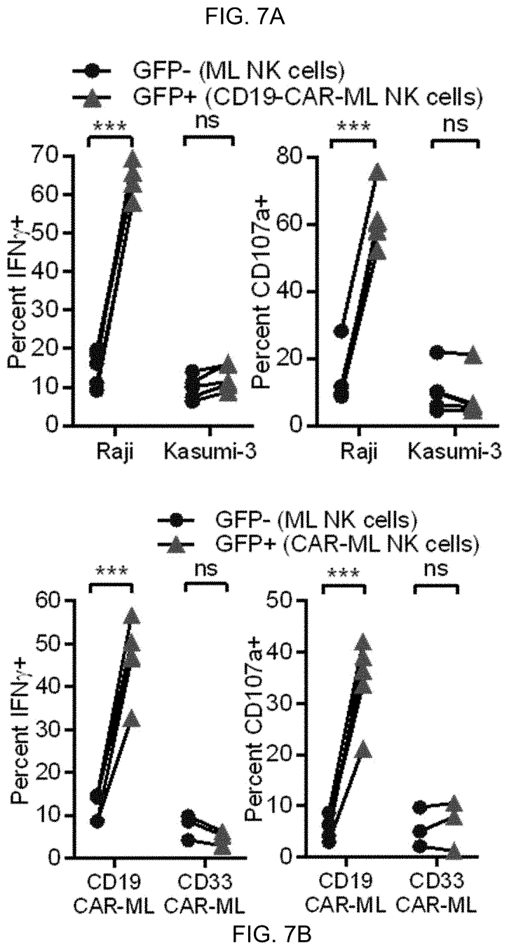

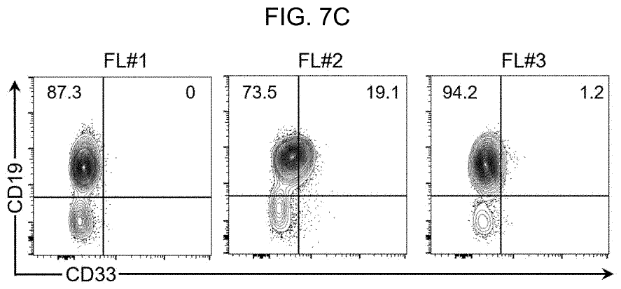

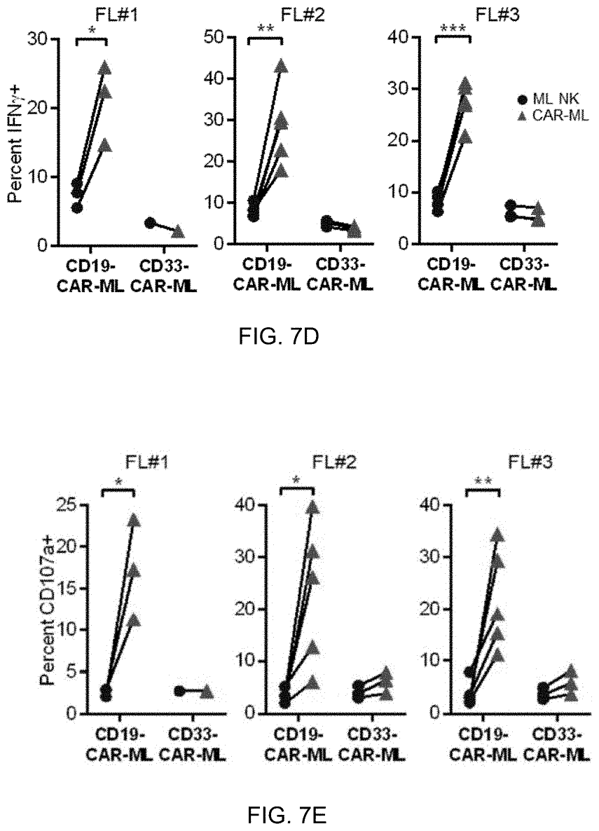

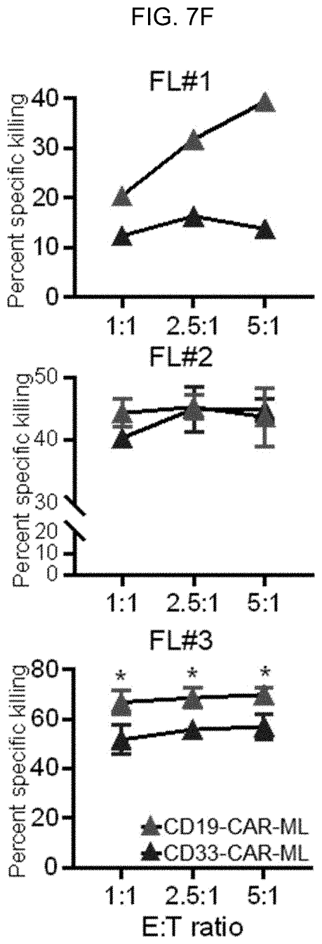

It was shown that CD19-CARML NK cells display antigen (CD19) specific enhanced responses against tumor cell lines and primary follicular lymphoma targets (see e.g., FIG. 7).

It was also shown that CD33-CARML NK cells display antigen (CD33) specific enhanced responses against tumor cell lines (see e.g., FIG. 9).

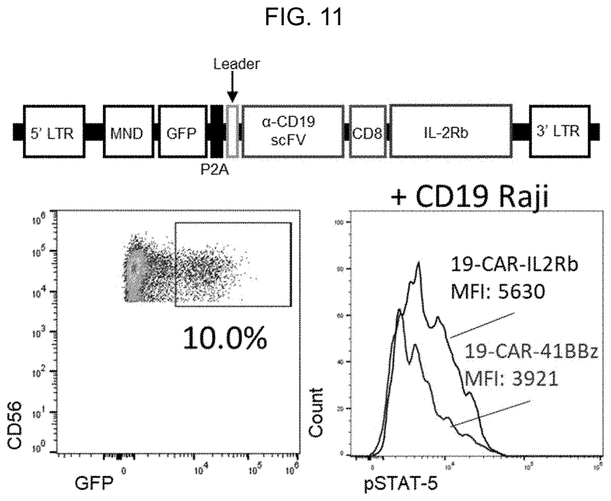

It was also shown that CD19-CAR (IL2Rb)-ML NK cells display enhanced pSTAT-5 signaling in the presence of CD19+ Raji targets (see e.g., FIG. 11).

NK Cells Expand In Vivo and Control Tumor Burden

This study demonstrated that CD19-specific ML NK cells expand in vivo and control tumor burden in CD19+ Raji bearing mice (see e.g., FIG. 8).

Strategy to Tailor CARML NK to Different Functions

Strategies for memory-like NK cell specific genetic modifications to optimize anti-tumor responses, cytokine production, cytotoxicity, proliferation, persistence are described in FIG. 4, FIG. 5, and FIG. 12. The rationale for designing CAR to specifically stimulate ML NK cells has been shown to be effective. For example, NKG2D expression is increased in ML NK cells, and incorporating the NKG2D TM domain results in enhanced signaling in CARML NK cells.

CARML Constructs

CD19, CD33, and CD123 CARML NK cells with various signaling domains have been generated using the protocols as described herein (see e.g., FIG. 1B, FIG. 6B, FIG. 8, FIG. 12).

Optimized Product Generation

Here, primary NK cells were purified from peripheral blood mononuclear cells (PBMCs). A PBMC is any peripheral blood cell having a round nucleus. These cells consist of lymphocytes (T cells, B cells, NK cells) and monocytes, whereas erythrocytes and platelets have no nuclei, and granulocytes (neutrophils, basophils, and eosinophils) have multi-lobed nuclei. Other products were derived from stem cells or cell lines.

It was discovered that the order of cytokine addition was important for ML NK cell production. On D-1 CIML activating cytokines, IL-12/15/18 were added. On DO cytokines were washed away and cells continued to culture in IL-15 for the remainder of the assay.

Here, the CAR was introduced using lentivirus. It was discovered that not just any viral construct will work and that CAR cannot be introduced to the ML NK cells using other viral particles currently in use for T cells.

It was also discovered that polybrene, used in other procedures killed the NK cells. As such, polybrene is not used during the transduction, which is conventionally used.

Construction of Lentiviral Vector and Transduction of NK Cells

The cassette encoding the chimeric antigen receptor (CAR) was incorporated into a MND lentiviral backbone to generate the lentiviral vector. To produce the lentiviral supernatant, 293T cells were cotransfected with lentiviral vectors, pMND-G, pMND-Lg, and pMDN-REV, using the Calcium Chloride transfection reagent. Supernatant containing the lentivirus was collected 24-48 and hours later and concentrated using ultracentrifugation. For transduction, purified cytokine activated (IL-12/15/18) NK cells were plated in complete culture media supplemented with 50 ng/mL IL-15. Viral supernatant was added to the cells, which are spinfected at 2000 rpm for 90 m at room temperature. The cells were incubated at 37° C. in 5% CO₂. To maximize viral transduction efficacy, cells were spinfected on days 1 and 2. The cells were then washed and used immediately or cultured in complete media supplemented with 1 ng/mL IL-15. Maximum vector expression is expected by day 7.

As described in FIG. 1A, FIG. 1B, FIG. 6A, and FIG. 6B, NK cells are purified from normal donor PBMC and incubated in IL-12/15/18. The cytokines are washed away and the cells are then incubated in high dose IL-15 and transduced with CAR lentivirus, 2×. Cells were allowed to rest (in vivo or in vitro) and assessed for enhanced effector functions.

### Example 2: Clinical Use of CARML NK

This example describes the clinical processing for treating patients with (A) haplo/allogeneic CARML NK cells or (B) autologous CARML NK cells (see e.g., FIG. 13).

Apheresis will be performed, NK cells purified and activated with IL-12/IL-15/IL-18 for about 12 hours. The NK cells will be washed and spinfected with CAR lentivirus, twice over about two days. The cells will be washed and infused into the patient at about 10⁷ cell/kg. In the haplo/allo setting the cells will be supported with rhIL-2 and in the autologous setting the cells will be supported with IL-15.

1. A chimeric antigen receptor (CAR) construct comprising:
(i) a targeting antibody fragment against a disease-associated antigen;
(ii) a transmembrane domain; and
(iii) at least one intracellular signaling domain,
wherein the CAR construct is capable of being expressed or functioning in a memory-like natural killer (ML NK) cell.

2. The CAR construct of claim 1, wherein the disease-associated antigen is selected from the group consisting of CD19, CD33, CD123, CD20, BCMA, Mesothelin, EGFR, CD3, CD4 BAFF-R, EGFR, HER2, gp120, or gp41.

3. The CAR construct of claim 1, wherein the transmembrane domain is selected from the group consisting of NKG2D, FcγRIIIa, NKp44, NKp30, NKp46, actKIR, NKG2C, CD8a, and IL15Rb.

4. The CAR construct of claim 1, wherein the at least one intracellular signaling domain is selected from the group consisting of CD137/41BB, DNAM-1, NKp80, 2B4, NTBA, CRACC, CD2, CD27, one or more integrins, IL-15R, IL-18R, IL-12R, IL-21R, IRE1a, and combinations thereof.

5. The CAR construct of claim 1, wherein the at least one intracellular signaling domain is a transmembrane adapter.

6. The CAR construct of claim 1, further comprising a transmembrane adapter or hinge.

7. The CAR construct of any one of claims 5 to 6, wherein the transmembrane adapter is selected from the group consisting of FceR1γ, CD3ζ, DAP12, DAP10, and combinations thereof.

8. The CAR construct of claim 4, wherein the one or more integrins are selected from the group consisting of ITGB1, ITGB2, ITGB3, and combinations thereof.

9. The CAR construct of claim 1, wherein the targeting antibody fragment against a disease-associated antigen comprises a scFv selected from the group consisting of:
(i) anti-CD19 scFv comprising an amino acid sequence of SEQ ID NO: 1;
(ii) anti-CD33 scFv comprising an amino acid sequence of SEQ ID NO: 2; and
(iii) anti-CD123 scFv comprising an amino acid sequence of SEQ ID NO: 3.

10. The CAR construct of claim 3, wherein the transmembrane domain is selected from the group consisting of:
NKG2D comprising an amino acid sequence of SEQ ID NO: 5;
FcγRIIIa comprising an amino acid sequence of SEQ ID NO: 7;
NKp44 comprising an amino acid sequence of SEQ ID NO: 9;
NKp30 comprising an amino acid sequence of SEQ ID NO: 11;
NKp46 comprising an amino acid sequence of SEQ ID NO: 13;
actKIR comprising an amino acid sequence of SEQ ID NO: 15;
NKG2C comprising an amino acid sequence of SEQ ID NO: 17;
CD8α comprising an amino acid sequence of SEQ ID NO: 19; and
IL15Rb comprising an amino acid sequence of SEQ ID NO: 21.

11. The CAR construct of claim 6, wherein the hinge is selected from the group consisting of:
NKG2D comprising an amino acid sequence of SEQ ID NO: 4;
FcγRIIIa comprising an amino acid sequence of SEQ ID NO: 6;
NKp44 comprising an amino acid sequence of SEQ ID NO: 8;
NKp30 comprising an amino acid sequence of SEQ ID NO: 10;
NKp46 comprising an amino acid sequence of SEQ ID NO: 12;
actKIR comprising an amino acid sequence of SEQ ID NO: 14;
NKG2C comprising an amino acid sequence of SEQ ID NO: 16;
CD8α comprising an amino acid sequence of SEQ ID NO: 18; and
IL15Rb comprising an amino acid sequence of SEQ ID NO: 20.

12. The CAR construct of claim 1, wherein the at least one intracellular signaling domain is selected from the group consisting of:
CD137/41BB comprising an amino acid sequence of SEQ ID NO: 22;
DNAM-1 comprising an amino acid sequence of SEQ ID NO: 23;
NKp80 comprising an amino acid sequence of SEQ ID NO: 24;
2B4 comprising an amino acid sequence of SEQ ID NO: 25;
NTBA comprising an amino acid sequence of SEQ ID NO: 26;
CRACC comprising an amino acid sequence of SEQ ID NO: 27;
CD2 comprising an amino acid sequence of SEQ ID NO: 28);
CD27 comprising an amino acid sequence of SEQ ID NO: 29);
integrins, ITGB1 comprising an amino acid sequence of SEQ ID NO: 30, ITGB2 comprising an amino acid sequence of SEQ ID NO: 31, or ITGB3 comprising an amino acid sequence of SEQ ID NO: 32;
IL15RB comprising an amino acid sequence of SEQ ID NO: 33;
IL18R comprising an amino acid sequence of SEQ ID NO: 34;
IL12R, IL12RB1 comprising an amino acid sequence of SEQ ID NO: 35 and IL12RB2 comprising an amino acid sequence of SEQ ID NO: 36;
IL21R comprising an amino acid sequence of SEQ ID NO: 37;
IRE1a comprising an amino acid sequence of SEQ ID NO: 38; and combinations thereof.

13. A memory-like natural killer (ML NK) cell comprising the CAR construct of claim 1.

14. A method of generating chimeric antigen receptor memory-like natural killer (CARML NK) cells comprising:
providing NK cells, activating cytokines comprising IL-12/15/18, and IL-15,
contacting the NK cells and activating cytokines for an amount of time sufficient to form cytokine-activated memory-like (ML) NK cells;
transducing a chimeric antigen receptor (CAR) via a viral vector into the cytokine-activated ML NK cells in the presence of IL-15 for an amount of time sufficient to virally transduce CAR into the cytokine-activated ML NK cells, resulting in CAR-transduced ML NK cells; and
incubating the CAR-transduced ML NK cells in the presence of IL-15 for an amount of time sufficient to form CAR-expressing ML NK (CARML NK cells).

15. The method of claim 14, wherein the NK cells were isolated from peripheral blood mononuclear cells (PBMCs).

16. The method of claim 14, wherein the amount of time sufficient to form cytokine-activated NK cells is between about 8 and about 24 hours, about 12 hours, or about 16 hours.

17. The method of claim 14, wherein the amount of time sufficient to virally transduce CAR into the ML NK cells is between about 12 hours and about 24 hours.

18. The method of claim 14, wherein the amount of time sufficient to form ML NK cells expressing CAR (CARML NK cells) is at least between about 3 days and about 8 days or about 7 days.

19. The method of claim 14, wherein the viral vector comprises a chimeric antigen receptor (CAR) is a CAR lentivirus.

20. The method of claim 14, wherein the viral vector is a lentiviral vector selected from the group consisting of pMND-G, pMND-Lg, pMDN-REV, and combinations thereof.

21. The method of claim 14, wherein transducing a chimeric antigen receptor (CAR) via a viral vector into the cytokine-activated ML NK cells is performed in the absence of polybrene.

22. A chimeric antigen receptor memory-like natural killer (CARML NK) cell made according to the method of any one of claims 14 to 21 comprising the CAR construct of any one of claims 1 to 13.

23. A method of inducing an immune response to a disease in a subject in need thereof comprising:
administering a chimeric antigen receptor memory like (CARML) NK cell to the subject,
wherein the CARML NK cell comprises a chimeric antigen receptor (CAR) comprising
(i) a targeting antibody fragment against a disease-associated antigen;
(ii) a transmembrane domain; and
(iii) at least one intracellular signaling domain.

24. The method of claim 23, wherein the disease-associated antigen is selected from the group consisting of CD19, CD33, CD123, CD20, BCMA, Mesothelin, EGFR, CD3, CD4 BAFF-R, EGFR, HER2, gp120, or gp41.

25. The method of claim 23, wherein the transmembrane domain is selected from the group consisting of NKG2D, FcγRIIIa, NKp44, NKp30, NKp46, actKIR, NKG2C, CD8a, and IL15Rb.

26. The method of claim 23, wherein the at least one intracellular signaling domain is selected from the group consisting of CD137/41BB, DNAM-1, NKp80, 2B4, NTBA, CRACC, CD2, CD27, one or more integrins, IL-15R, IL-18R, IL-12R, IL-21R, IRE1a, and combinations thereof.

27. The method of claim 23, wherein the at least one intracellular signaling domain is a transmembrane adapter.

28. The method of claim 23, further comprising a transmembrane adapter.

29. The method of any one of claims 27 to 28, wherein the transmembrane adapter is selected from the group consisting of FceR1γ, CD3ζ, DAP12, DAP10, and combinations thereof.

30. The method of claim 26, wherein the one or more integrins are selected from the group consisting of ITGB1, ITGB2, ITGB3, and combinations thereof.

31. The method of claim 23, the targeting antibody fragment against a disease-associated antigen comprises a scFv selected from the group consisting of:
(i) anti-CD19 scFv comprising an amino acid sequence of SEQ ID NO: 1;
(ii) anti-CD33 scFv comprising an amino acid sequence of SEQ ID NO: 2; and
(iii) anti-CD123 scFv comprising an amino acid sequence of SEQ ID NO: 3.

32. The method of claim 25, wherein the transmembrane domain is selected from the group consisting of:
NKG2D comprising an amino acid sequence of SEQ ID NO: 5;
FcγRIIIa comprising an amino acid sequence of SEQ ID NO: 7;
NKp44 comprising an amino acid sequence of SEQ ID NO: 9;
NKp30 comprising an amino acid sequence of SEQ ID NO: 11;
NKp46 comprising an amino acid sequence of SEQ ID NO: 13;
actKIR comprising an amino acid sequence of SEQ ID NO: 15;
NKG2C comprising an amino acid sequence of SEQ ID NO: 17;
CD8α comprising an amino acid sequence of SEQ ID NO: 19; and
IL15Rb comprising an amino acid sequence of SEQ ID NO: 21.

33. The method of claim 23, further comprising a hinge selected from the group consisting of:
NKG2D comprising an amino acid sequence of SEQ ID NO: 4;
FcγRIIIa comprising an amino acid sequence of SEQ ID NO: 6;
NKp44 comprising an amino acid sequence of SEQ ID NO: 8;
NKp30 comprising an amino acid sequence of SEQ ID NO: 11;
NKp46 comprising an amino acid sequence of SEQ ID NO: 12;
actKIR comprising an amino acid sequence of SEQ ID NO: 14;
NKG2C comprising an amino acid sequence of SEQ ID NO: 16;
CD8α comprising an amino acid sequence of SEQ ID NO: 18; and
IL15Rb comprising an amino acid sequence of SEQ ID NO: 20.

34. The method of claim 23, wherein the at least one intracellular signaling domain is selected from the group consisting of:
CD137/41BB comprising an amino acid sequence of SEQ ID NO: 22;
DNAM-1 comprising an amino acid sequence of SEQ ID NO: 23;
NKp80 comprising an amino acid sequence of SEQ ID NO: 24;
2B4 comprising an amino acid sequence of SEQ ID NO: 25;
NTBA comprising an amino acid sequence of SEQ ID NO: 26;
CRACC comprising an amino acid sequence of SEQ ID NO: 27;
CD2 comprising an amino acid sequence of SEQ ID NO: 28);
CD27 comprising an amino acid sequence of SEQ ID NO: 29);
integrins, ITGB1 comprising an amino acid sequence of SEQ ID NO: 30, ITGB2 comprising an amino acid sequence of SEQ ID NO: 31, and ITGB3 comprising an amino acid sequence of SEQ ID NO: 32;
IL15RB comprising an amino acid sequence of SEQ ID NO: 33;
IL18R comprising an amino acid sequence of SEQ ID NO: 34;
IL12R, IL12RB1 comprising an amino acid sequence of SEQ ID NO: 35 and IL12RB2 comprising an amino acid sequence of SEQ ID NO: 36;
IL21R comprising an amino acid sequence of SEQ ID NO: 37;
IRE1a comprising an amino acid sequence of SEQ ID NO: 38; and combinations thereof.

35. The method of claim 23, wherein the CAR construct is capable of expressing or functioning in a memory-like natural killer (ML NK) cell.

36. The method of claim 23, wherein the CARML NK cell induces an immune response to an antigen-specific target.

37. The method of claim 23, wherein the CARML NK cell reduces tumor burden.

38. The method of claim 23, wherein the targeting antibody fragment against a disease-associated antigen comprises a single chain variable fragment (scFv) against a disease-associated antigen.

39. The method of claim 23, wherein the subject has a disease having a disease-associated antigen.

40. The method of claim 23, wherein the antigen is a B cell antigen and the disease is selected from the group consisting of a hematological cancer, an autoimmune disease, and immune system disorders.

41. The method of claim 23, wherein the antigen is a tumor-associated antigen (TAA) and the disease is cancer.

42. The method of claim 23, wherein the CARML NK cell has an enhanced functional response against antigen targets or epitopes compared to a control.

43. The method of claim 42, wherein the control is an ML NK cell without CAR, an MLNK cell without a scFv, an NK cell with CAR, an NK cell with CAR scFv, an ML NK comprising a scFv not associated with a target, or NK comprising a scFv not associated with a target.

44. The method of claim 23, wherein the subject has cancer, an autoimmune condition, or an infectious disease (e.g., bacterial, viral).

45. A method of administering CARML NK cells to a subject in need thereof comprising:
isolating NK cells from a subject or a donor;
generating CARML NK cells according to claim 14; and
administering a therapeutically effective amount of CARML NK cells into the subject.

46. The method of claim 45, wherein the therapeutically effective amount of CARML NK cells is about 10⁷ cell/kg.

47. The method of claim 45, wherein rhIL-2 or IL-15 is administered to the subject.

48. A chimeric antigen receptor (CAR) construct comprising:
(i) an anti-CD19 scFv comprising SEQ ID NO: 1, an anti-CD33 scFv comprising SEQ ID NO: 2, or an anti-CD123 scFv comprising SEQ ID NO: 3;
(ii) a CD8α transmembrane domain comprising SEQ ID NO: 19, a NKp30 transmembrane domain comprising SEQ ID NO: 11, or a NKG2D transmembrane domain comprising SEQ ID NO: 5; and
(iii) a CD137 intracellular signaling domain comprising SEQ ID NO: 22, a IL-15R intracellular signaling domain comprising SEQ ID NO: 33, or a 2B4 intracellular signaling domain comprising SEQ ID NO: 25;
wherein the CAR construct is capable of being expressed or functioning in a memory-like natural killer (ML NK) cell.
